(19) United States
(12) Patent Application Publication
(10) Pub. No.: US 2022/0117508 A1
(43) Pub. Date: Apr. 21, 2022

(54) METHODS FOR ACCURATE NEEDLE-FREE ASSESSMENT OF MYOCARDIAL OXYGENATION

(71) Applicant: Cedars-Sinai Medical Center, Los Angeles, CA (US)

(72) Inventors: Rohan DHARMAKUMAR, Moorpark, CA (US); Hsin-Jung YANG, West Hollywood, CA (US)

(73) Assignee: Cedars-Sinai Medical Center, Los Angeles, CA (US)

(21) Appl. No.: 17/428,011

(22) PCT Filed: Feb. 7, 2020
(86) PCT No.: PCT/US2020/017320

§ 371 Date: Aug. 3, 2021

Related U.S. Application Data
(60) Provisional application No. 62/802,682, filed on Feb. 7, 2019.

(51) Int. Cl. A61B 5/055; A61B 5/026; A61B 5/145; A61B 5/0295; A61B 5/00; G06T 7/00; G06T 7/11; G01R 33/56; G01R 33/561

(52) U.S. Cl. CPC A61B 5/055; A61B 5/0263; A61B 5/14542; A61B 5/0295; A61B 5/4884; G06T 7/0014; G06T 2207/30048; G01R 33/5608; G01R 33/561; A61B 2576/00; G06T 2207/10088; G06T 2207/30104; G06T 7/11

(57) ABSTRACT

Described herein are methods for cardiovascular imaging for diagnosing and/or detecting various cardiovascular diseases. Various embodiments of the invention provide using magnetic resonance imaging of the cardiovascular system of a subject at rest or a normocapnic condition, as well as at a stressed or hypercapnic condition, in a repeated manner enhancing the statistical power, such that fast, motion-corrected, free-breathing, whole-heart imaging of the cardiovascular system is utilized to identify impaired cardiovascular function in a manner with improved specificity and accuracy.

### CROSS-REFERENCE TO RELATED APPLICATION

This application includes a claim of priority under 35 U.S.C. § 119(e) to U.S. provisional patent application No. 62/802,682, filed Feb. 7, 2019, the entirety of which is hereby incorporated by reference.

### STATEMENT REGARDING FEDERALLY SPONSORED RESEARCH OR DEVELOPMENT

This invention was made with government support under Grant No. HL091989 awarded by National Institutes of Health. The government has certain rights in the invention.

### FIELD OF THE INVENTION

The invention relates to cardiovascular imaging for diagnosing and/or detecting various cardiovascular diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Ischemic heart disease (IHD) is the leading cause of death in the Western world. It often stems from atherosclerotic narrowing of the coronary arteries (stenosis), leading to reduced blood flow and oxygen supplied to the heart muscle (myocardium). This causes myocardial ischemia during physical exertion, a condition where the oxygen supply to the heart muscle does not meet the myocardial oxygen demand. Ischemic burden (the extent and severity of ischemia) is a key predictor of major adverse cardiac events (MACE), including stroke, heart attack (myocardial infarction) and death. Early interventions (medical, surgical or lifestyle), guided by cut-offs based on ischemic burden, are crucial for reducing MACE in IHD patients.

The capability of blood vessels to supply the heart muscle (myocardium) with oxygen, called myocardial oxygenation, is an important determinant of cardiac function. Impairment of myocardial oxygenation is a defining feature of ischemic heart disease (IHD), which afflicts millions of people around the world. DID is caused by pathological conditions that affect the blood vessels supplying oxygen to the heart muscle. Methods for detecting myocardial oxygenation deviations from the norm can be sought after to guide interventions (medical, surgical or lifestyle) and to prevent acute life-threatening events such as heart attacks (myocardial infarction).

Long-established (single-photon emission computed tomography, SPECT, or positron emission tomography, PET) and more recently developed (e.g., first-pass perfusion cardiac magnetic resonance) techniques are used to gather information on ischemic burden in lieu of invasive cardiac catheterization.

For example, with magnetic resonance imaging (MRI), when a substance such as human tissue is subjected to a uniform magnetic field (polarizing field B₀), the individual magnetic moments of the excited nuclei in the tissue attempt to align with this polarizing field, but precess about it in random order at their characteristic Larmor frequency. If the substance, or tissue, is subjected to a magnetic field (excitation field B₁) that is in the x-y plane and that is near the Larmor frequency, the net aligned moment, M_{z}, may be rotated, or “tipped”, into the x-y plane to produce a net transverse magnetic moment Mₜ. A signal is emitted by the excited nuclei or “spins”, after the excitation signal B₁ is terminated, and this signal may be received and processed to form an image. Magnetic relaxation properties of tissues give rise to contrast in MR images. Variations in tissue relaxation times help distinguish the healthy and the pathological states.

Once a main magnetic field of the scanner (B₀) is selected, additional, smaller magnetic fields with changing electrical fields are added. Each magnetic resonance (MR) scanner has 3 sets of spatial encoding electrical coils to produce magnetic fields in the x, y, and z directions. These coils can be adjusted to produce not a constant field but a gradient, in other words a magnetic field that changes in strength depending on your position. These magnetic fields are much weaker than B₀ and vary linearly across the x, y, or z direction. They can even be turned on in combinations to create a linear gradient in any arbitrary direction in space. By the Larmor equation, ƒ=γ*B, so that if the magnetic field varies across space, the precession frequency of the protons will vary as well. Hence, with gradients, one can separate different parts of anatomy by frequency. For example, magnetic field gradients can be closer to the order of several hundredths of a percent over a couple centimeters

Protons exchange energy efficiently if the frequency of the energy matches their precession frequency. Thus, the 90-degree and 180-degree pulses are typically sent at the Larmor frequency of the proton. By turning on the magnetic field gradient, the protons at each position in the body experience a slightly different magnetic field—slightly more or less than B₀. Thus, one gets a gradient of precession frequencies along the body that differ. By then altering the frequency of the 90-degree and 180-degree pulses, one can excite different protons. For example, a scanner can select a particular slice to image by turning on the slice-select gradient and then altering the frequency of the excitation pulses (90, 180, and any inversion pulse) to match the frequency at the desired slice position. Protons not in the slice will not get excited since their Larmor frequency will not match the frequency of the pulse, thus they won't efficiently receive energy from the pulse. The slice-select gradient doesn't have to be on during readout since no protons are being excited then—although the readout frequency will be centered at the particular frequency of that slice.

The MR scanner will receive the entire signal from that slice under a slice-select gradient, and Fourier transform can be used to split up signals from that slice by frequency. The MR scanner acquires (listens to, samples) the signal over time, and the signal is digitized and stored in the computer, in a matrix known as K-space. The Fourier transform of K-space is real space—the image.

To localize a proton to a slice and then along one axis within the slice relies on changing the frequency of the protons. In order to localize along a third axis, the phase of the protons is manipulated. To do that, one turns on a gradient for a short amount of time, then turns it off. Once the gradient is off, the magnetic field strength B returns to its normal level (for the slice)—and so all of the protons go back to precessing at the exact same frequency. However, while the gradient was on, the protons were moving faster than their neighbors, so when the gradient is turned off they're still ahead—they have accumulated a phase shift. For example, one can create phase shifts that vary across the y-axis by transiently turning on a gradient along that direction. In order to use both phase and frequency encoding, one can first turn on the phase encoding gradient, then turn it off; this happens during the time of waiting for the echo. At the time of the echo, one can turn on the frequency-encoding gradient only. This changes the frequencies of the protons while their precessions are being measured—they still ‘remember’ their phase shifts from earlier. As one cannot measure more than one phase per frequency, one way to get around that is to perform many different, separate phase-encoding gradients, and use the Fourier transform to figure out each phases. One would need to repeat an entire MR sequence for each different phase-encoding gradient to be measured, and do one for each row of pixels in an image. For example, for an image of 256×256, one would need to do 256 phase-encoding steps. Each phase-encoding step is stored as a different row in the K-space matrix. By performing a 2-dimensional Fourier transform (i.e., applying the Fourier transform in both directions), one can analyze both frequency and phase—and obtain the actual image.

MR images are acquired in K-Space, where image information is stored in the frequency domain rather than the spatial domain. K-space is a matrix the same size as the resulting image. The points in K-space are acquired through frequency encoding and successive phase encoding steps. Once the entire matrix is filled in, the inverse Fourier transform decodes the frequency information into the actual image. A traditional way of acquiring K-space data is through Cartesian, or rectilinear, phase and frequency encoding. This fills the K-space matrix in successive lines, where each line of K-space is a separate phase encoding step (the phase encoding may be done in any arbitrary axis of a body). There are several disadvantages of Cartesian filling. For example, the center of K-space, which corresponds to image contrast, is only acquired once. Also, each line is acquired only once and is therefore susceptible to patient motion. Finally, one must acquire enough phase encoding steps to prevent aliasing (or wrap) artifact from body parts outside the desired field of view. An alternative to a Cartesain trajectory is radial filling, where the scanner acquires multiple radial lines (like a starburst) to fill the K-space matrix. A radial trajectory has no phase encoding steps; each line in the trajectory is a frequency encoding step. Thus, there is no possibility of wrap artifact with radial trajectories. This allows small field-of-view (FOV) imaging. Also note that the center of K-space is sampled multiple times, although the periphery is relatively undersampled. This may compromise spatial resolution. However, radial sampling tends to be much less sensitive to motion artifacts. The radial lines that are acquired must be “gridded” or interpolated to form a standard Cartesian grid before application of the inverse Fourier transform.

Yet conventional MRI, SPECT or PET techniques generally have known limitations. They expose the patients to either ionizing radiation or exogenous contrast agents, both of which carry significant risks. For example, this is a pressing problem for end-stage renal disease (ESRD) patients in whom active surveillance for IHD is needed to manage the >10-fold cardiovascular mortality in this patient population compared to the general population; yet frequent ischemia testing is contraindicated because contrast agents are needed or patients are exposed to exorbitant ionizing radiation. Furthermore, the current diagnosis of IHD largely relies on surrogate metrics, notably electrocardiography (ECG), myocardial blood flow (MBF) and myocardial blood volume, which are either non-specific or operator dependent. For example, ECG may be non-specific in that it cannot identify asymptomatic patients with significant coronary stenosis unless combined with exercise stress, which is not tolerated by more than 50% of IHD patients. MBF changes can be determined using SPECT, PET, first-pass perfusion MRI and contrast-enhanced echocardiography, where SPECT and PET approaches require radioactive tracers and first-pass perfusion MRI requires exogenous contrast media based on gadolinium. Oxygen supply and demand to a given physiological stimulus are variable in every patient, measuring MBF or indexing myocardial blood volume may not provide full physiological insight into the extent and severity of myocardial ischemia in patients with IHD.

Blood oxygen level-dependent (BOLD) cardiac MRI (CMR) is a potentially safe alternative because it is free of ionizing radiation or the use of an exogenous contrast agent. BOLD CMR depicts changes in regional blood concentrations of oxy- and deoxy-hemoglobin, depending on tissue microvasculature and resulting blood volume. Without being bound by a theory, oxyhemoglobin has no unpaired electrons and is weakly diamagnetic; and whereas when oxygen is released to form deoxyhemoglobin, four unpaired electrons are exposed at each iron center, causing the molecule to become strongly paramagnetic, which shortens T2 and T2*. Increases in O₂ saturation increase the BOLD imaging signal (T2 or T2*), whereas decreases diminish it.

However, the reliability (sensitivity, specificity and accuracy) of BOLD CMR has remained a major weakness when compared to established clinical standards such as PET. It is not clear whether this discrepancy is between a test for myocardial oxygenation and a test for myocardial blood flow or whether it is a consequence of known accuracy limitations of BOLD-CMR. Uncertainty of a measurement is fundamentally determined by noise. In BOLD-MRI, physiological noise (motion) and imaging noise (limitations in signal-reception elements, termed “RF coils”) dominate the small signal changes that result from oxygenation level changes. This makes it challenging to accurately index an observed signal change against blood oxygenation. Although injectable drugs (e.g., adenosine) can stimulate changes in the heart, they cannot be repeatedly administered within a same examination due to adverse side effects; and conventional BOLD-CMR is essentially two-dimensional, which is typically limited to a single slice, and current acquisition schemes are subject to heart-rate variations in between different vasodilatory states, making it difficult to unmask “true” signals associated with physiological changes in blood flow from the readouts. Physiological and imaging noises during CMR data acquisition persistently limited the ability to detect small changes in oxygenation-sensitive CMR signals in the heart, which impeding its clinical adoption. Thus there is a need in the art for non-invasive cardiovascular imaging methods that overcome the limitations of known methods.

### SUMMARY

The following embodiments and aspects thereof are described and illustrated in conjunction with systems, articles of manufacture, compositions and methods which are meant to be exemplary and illustrative, not limiting in scope.

Various embodiments of the invention provide method for performing a cardiac stress testing, detecting the presence of or determining the progression of a cardiovascular disease, and/or assessing the risk of developing the cardiovascular disease in a subject, using a magnetic resonance imaging (MRI) system, the method comprising: (a) administering a stress agent to the subject over one or more periods of time in one or more amounts, wherein at least one amount is effective for increasing blood velocity and/or flow rate at the cardiovascular system of the subject; (b) directing the MRI system to perform a sequence that is sensitive to blood oxygenation, blood volume, and/or blood flow at the cardiovascular system of the subject to acquire a plurality of MR data sets corresponding to a plurality of MR acquisitions, wherein the plurality of MR acquisitions comprises one or more acquisitions whose MR data set is acquired during the one or more periods of time when the at least one effective amount of the stress agent is administered to the subject, and one or more acquisitions whose MR data set is acquired when the subject is at rest or during administration of a different amount of the stress agent to the subject; and (c) reconstructing a series of images from each MR data set and registering the series of images to obtain a motion-corrected image for each MR acquisition, thereby obtaining a plurality of motion-corrected images corresponding to the plurality of MR acquisitions. Further aspects of these methods include an additional step (d) comparing the plurality of motion-corrected images in terms of image voxels or pixels characteristic of blood oxygenation, blood volume, or blood flow in at least one region of the cardiovascular system, wherein an absence of a statistically significant difference in the image voxels or pixels in at least one region of the cardiovascular system compared among the motion-corrected images acquired during the administration of the effective amount of the stress agent and those acquired when the subject is at rest or during the administration of the different amount of the stress agent is indicative of impaired blood oxygenation, impaired blood volume, or impaired blood flow, respectively, in the at least one region of the cardiovascular system of the subject

Some embodiments of the methods for performing a cardiac stress testing, detecting the presence of or determining the progression of a cardiovascular disease, and/or assessing the risk of developing the cardiovascular disease in a subject using an MRI system, include a blockwise stressing for MRI acquisitions, in steps such as: (a) directing the MRI system to perform a first sequence that is sensitive to blood oxygenation, blood volume, and/or blood flow, at a cardiovascular system of the subject in a rest state or at a pre-defined baseline/reference partial pressure of arterial carbon dioxide (PaCO₂) level to acquire a first set of reference MR data from the subject; (b) administering an effective amount of a stress agent, e.g., selected from the group consisting of CO₂ and an admixture comprising CO₂, wherein the stress agent is administered in an amount effective for inducing hyperemia or causing increased blood velocity or flow rate in the subject; (c) directing the MRI system to perform a second sequence that is sensitive to the blood oxygenation, the blood volume, and/or the blood flow at the cardiovascular system of the subject at an attained stress PaCO₂ level or while the stress agent is administered according to step (b) to acquire a first set of stress MR data from the subject; (d) repeating steps (a)-(c) at least once, each repetition generating a subsequent set of reference MR data and a subsequent set of stress MR data; (e) reconstructing a series of reference images from each set of the reference MR data and registering the series of reference images to obtain a motion-corrected reference image for each set of the reference MR data, thereby obtaining a plurality of motion-corrected reference images from repetition of step (a), and reconstructing a series of stress images from each set of the stress MR data and registering the series of stress images to obtain a motion-corrected stress image for each set of the stress MR data, thereby obtaining a plurality of motion-corrected stress images from repetition of steps (b) and (c); and (f) comparing the plurality of motion corrected reference images and the plurality of motion corrected stress images in terms of image voxels or pixels characteristic of the blood oxygenation, the blood volume, and/or the blood flow in at least one region of the cardiovascular system, wherein an absence of a statistically significant difference in the image voxels or pixels between the plurality of motion corrected reference images and the plurality of motion corrected stress images is indicative of impaired blood oxygenation, impaired blood volume, and/or impaired blood flow, respectively, in the at least one region of the cardiovascular system of the subject.

Other embodiments of the methods for performing a cardiac stress testing, detecting the presence of or determining the progression of a cardiovascular disease, and/or assessing the risk of developing the cardiovascular disease in a subject using an MRI system, include an increasing amount (e.g., stepwise) of stressing for multiple MRI acquisitions at different levels of stressing, wherein the statistical analysis showing the subject's blood oxygenation, blood volume or blood flow does not change due to different levels of stressing, i.e., absence of a statistical significant difference among MRI voxels/pixels obtained while the subject is at different levels of stressing, is indicative that the subject's cardiovascular system has impaired function.

Further embodiments of the invention provide the methods include the first sequence, the second sequence, or both, which include one or more of a saturation recovery (SR) pulse, a navigator pulse, an adiabatic pulse, a spoiled gradient-echo (GRE) pulse, a balanced steady-state free precessing (bSSFP) sequence, and compressive sampling. In some aspects, the saturation recovery pulse has a constant saturation recovery time. In some aspects, the sequences are a free-breathing three-dimensional (3D) T2-based sequence at a magnetic field of 3T or greater.

Further embodiments provide the methods additionally include segmenting the cardiovascular system in each motion-corrected reference image and each motion-corrected stress images, and wherein the step of comparing compares at least one segment of the cardiovascular system in the plurality of the motion-corrected reference images with the corresponding segment in the plurality of the motion-corrected stress images, wherein the lack of a statistically significant difference in the image voxels or pixels is indicative of the impaired blood volume, the impaired blood flow, and/or the impaired blood oxygenation in the at least one segment of the cardiovascular system.

In some embodiments, the MRI sequences are selected from T1, T2, T2*, and ASL. In some embodiments, the MRI sequences are BOLD-MRI, wherein the BOLD-MRI comprises a free-breathing 3D T2-based sequence at a magnetic field of at least 1.5T, 3T or greater.

In some embodiments, the step of comparing the plurality of motion corrected reference images and motion corrected stress images compares blood flow in at least one region/segment of the heart at the reference and stress arterial blood levels of CO₂. In some aspects, blood flow is determined by blood oxygenation in the region of the heart.

In some embodiments, the baseline/reference arterial blood level of carbon dioxide (CO₂) is for the subject a rest arterial blood level of CO₂. In some embodiments, the subject at rest is in a normocapnic state. In some embodiments, a pre-defined baseline PaCO₂ is about 35 mm Hg, 30 mm Hg, or 25 mm Hg.

In some embodiments, a generalized linear model statistical framework is used to compare among the plurality of motion-corrected images acquired while the subject is under no stress agent or at varying amounts of stress agents. Further embodiments of the statistical framework require a statistical power of at least 0.8 based on the plurality of motion-corrected images. In one embodiment, an anova statistical framewok with a statistical power of 0.8 or greater is used in the methods.

In some embodiments, the subject is in a hypercapnic state after administration of the stress agent. In some embodiments, the stress agent is CO₂ or admixture comprising CO₂, and is administered via inhalation. In some embodiments, an effective amount of the stress agent is administered to induce hyperemic response in the subject, e.g., by increasing the PaCO₂ in the subject to about 60 mmHg, about 65 mmHg, about 55 mmHg, or about 70 mmHg, while PaO₂ level is maintained at a level ranging from 50 mmHg to 150 mmHg.

In some embodiments, the cardiovascular disease being diagnosed or detected in one or more of the methods is selected from infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, coronary stenosis, LAD stenosis, and peripheral artery disease.

In some embodiments, the cardiovascular system being tested, diagnosed or examined in one or more of the methods comprises a heart. In some embodiments, the heart is selected from a whole heart, a portion of the heart, and a section of the heart. In some embodiments, the cardiovascular system comprises a myocardium. In some embodiments, the cardiovascular system comprises at least one coronary artery. In some embodiments, the blood volume is myocardial blood volume; the blood oxygenation is myocardial blood oxygenation; the blood flow is myocardial blood flow.

Computer readable storage mediums containing computer executable instructions for one or more of the methods are also provided.

Statistical parametric maps calculated from the p-value in one or more of the methods are also provided.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-1D depicts repeat stimulations and image averaging for enhancing myocardial 2D BOLD response. FIG. 1A shows the trace of achieved PaCO₂ levels during the scans. Healthy Animal without Coronary Stenosis: FIG. 1B shows a single-slice (2D) segmental BOLD response according to AHA segments 1 through 6 in a healthy animal (i.e., without coronary stenosis) during the four blocks of intermittent hypercapnia (4 stimulations), which highlights the dynamic signal response during the repeated stimulations. Animal with Coronary Stenosis: FIG. 1C shows that segmental BOLD response across AHA segments 1 through 6 during four blocks of intermittent hypercapnia (4 stimulations) from an animal with significant LAD coronary stenosis. FIG. 1D shows the 2D spatial maps of the BOLD response in the mid-ventricular myocardium after one hypercapnic stimulation (“single pair” of normocapnic and hypercapnic), on the left, the spatial map of the average BOLD response following 4 hypercapnic stimulations (“four pairs”), in the middle, and the ¹³N-ammonia PET response (myocardial perfusion reserve) which was acquired simultaneously with BOLD-MRI.

FIG. 2 depicts theoretical basis for objective assessment of myocardial BOLD Response. FIG. 2 shows the relation between BOLD response (vertical axis) and the number of stimulations (horizontal axis) required to establish statistical significance (color-coded p-values). For a given BOLD response, the number stimulations required for reliable assessment (p<0.05) of a change from baseline condition lies at the right of the white dotted line. For example, to reliably detect a BOLD response with peak BOLD signal response of 10%, greater than 3 measurements are needed. The color bar on the right provides the scale for p values associated with statistical significance.

FIG. 3A-3D depict cardiac fMRI framework integrating MRI, hypercapnic stimulation and statistical analysis. FIG. 3A describes data acquisition framework: The approach used to acquire 3D MRI under periodic changes in PaCO₂ (normocapnic and hypercapnic conditions), preceded by a short-delay (stabilization period) to ensure that the acquisitions are only triggered once the desired PaCO₂ are reached. FIG. 3B describes an exemplary time-efficient, free-breathing, confounder-corrected whole-heart T₂ mapping: The timing diagram and data encoding strategy are illustrated. The upper panel shows a T₂ preparation scheme composed of composite adiabatic RF pulses and spoiled gradient echo (GRE) readout are used to minimize B₁ and B₀ artifacts at 3T. A saturation-recovery (SR) preparation was added to eliminate the signal dependence on heart rate between segmented readouts and navigator pulses (NAV) were added to monitor the respiratory motion during acquisition. From “R” indicating ECG signal from heart to the end of GRE readout, as shown in the upper panel, refers to a timeline in one heart beat (between two consecutive heart beats). On the right of the upper panel shows the centric-encoding scheme with hybrid trajectory to ensure optimal T₂ weighting. The lower panel shows the motion-correction algorithm and T₂ mapping using a log-transformed linear least-squares fit. FIG. 3C describes 3D Myocardial BOLD Response: 3D T₂ maps (basal, mid-ventricular, and apical) acquired during normocapnia and hypercapnia (single stimulation block). For reference, results from 2D imaging obtained from a mid-ventricular slice are also shown. BOLD Response (computed as (hypercapnic myocardial T₂÷normocapnia myocardial T₂)×100%) for the 2D and 3D cases are shown on the top panel. FIG. 3D describes statistical framework: a schematic of the statistical framework employing repeated measures with one-way ANOVA to discriminate between myocardial segments that are statistically responsive and not, based on the hypothesis testing outlined in Example 1, following each repeat hypercapnic/normocapnic stimulation. The polar maps on the lower row show the AHA segmentation with p-values assigned on the statistical test.

FIG. 4A-4C depict application of cardiac fMRI approach for reliable identification of healthy myocardium. FIG. 4A describes Myocardial Statistical Parametric Mapping (SPM): Long- and short-axis volume rendered views of the heart with intensities denoting segmental p-values derived from the statistical framework from a typical healthy animal are shown. The polar maps at the bottom of the panel provide a bull's eye plot of p-values. FIG. 4B describes Myocardial SPM vs. ¹³N-Ammonia PET in Representative Case: the left side shows the mean and standard deviation of p-values across all segments for the case in FIG. 4A as a function of number of stimulation blocks (one through four). The right side of FIG. 4B shows the corresponding ¹³N-Ammonia PET MPR. FIG. 4C describes Myocardial SPM vs. ¹³N-Ammonia PET Across all Animals: The left side shows the average response across all animals and all myocardial segments following one and four stimulations, and the right side shows mean and scatter of MPR across all animals in response to hypercapnia.

FIG. 5A-5D depict cardiac fMRI based SPM for accurate identification of myocardial segments subtended by clinically significant coronary stenosis. FIG. 5A depicts myocardial SPM Under Coronary Stenosis: Long- and short-axis volume rendered views of the heart with intensities denoting segmental p-values derived from the statistical framework from one dog with clinically significant coronary stenosis is shown. The polar maps at the bottom of the panel provide p-values for the AHA segments. FIG. 5B depicts myocardial SPM vs. ¹³N-Ammonia PET (for a representative case): Left panel shows the mean and standard deviation of p-values across affected and remote segments for the case in FIG. 5A as a function of number of stimulation blocks (one through four); right panel shows the corresponding ¹³N-Ammonia PET MPR. FIG. 5C depicts myocardial SPM vs. ¹³N-Ammonia PET (for all cases): left panel shows the average response across all animals in the affected and remote myocardial segments following one and four stimulations; right panel shows the mean and scatter of PET-MPR across all animals in the remote and affected segments following hypercapnia. FIG. 5D depicts the results from sensitivity, specificity and accuracy determined following each stimulation (with PET serving as the ground truth).

FIG. 6 depicts an exemplary imaging study protocol for acquiring both PET and CMR images in an interleaved fashion. Two PET acquisitions (hypercapnic and normocapnic) were performed before and after the CMR BOLD acquisition and the order of the PET scans were randomized. Following the first PET scan, BOLD CMR acquisitions were prescribed in synchrony with the hypercapnia and normocapnia stimulation pairs. Respective 2D or 3D BOLD sequences were prescribed for each group of animals. Following CMR acquisitions, a second PET scan was acquired. A minimum of 50 minutes time delay after the first PET scan was introduced to allow sufficient decay of the isotope. In the stenosis studies, LAD occlusions were induced before the first image acquisition.

FIGS. 7A and 7B depict computer simulations and ex-vivo experiments. FIG. 7A shows simulated and experimental estimates of myocardial T2 values. Myocardial T2 derived from conventional T2 mapping sequence obtained at resting heartrate (60 bpm) was used as the ground truth. Computer simulations of the conventional 2D sequence shows slight T2 decrease with increasing heart rates. However, experimental values demonstrate the T2 reductions are sufficiently small and all T2 values acquired within the range of reasonable physiological conditions were not different from the resting T2 (all p=1). Computer simulation of the proposed sequence, on the other hand, shows accurate T2 measurement with no heart rate dependency and is in alignment with the experimental results. Myocardial T2 acquired under the different heart rates demonstrate no difference from the standard value (all p=1). FIG. 7B shows a representative set of mid-ventricular short-axis T2 maps using the different T2 mapping strategies and different heart rates are presented. All myocardial T2 maps showed T2 values that were not different from the T2 values acquired using conventional sequence at a representative resting heart rate of 60 bpm.

FIG. 8A-8C depict 9 Heart-rate independent T2 BOLD validated against 13N-PET in healthy canines under rest and stress. Note the concordance in myocardial T2 (FIG. 8A) and 13N-PET blood flow (FIG. 8B) and dependence of DHR on loss of BOLD contrast (FIG. 8C).

FIG. 9A-9C depict contrast-to-noise ratio (CNR) of native T2-w GRASP CMR. FIG. 9A: Effect of undersampling (no compressed sensing, “no CS”) and with compressed sensing (“CS”) at rest and at hyperemia with TRes of 11 heart beats. FIG. 9B: CNR dependence on TRes of GRASP MRI and CS. FIG. 9C: signal profile of blood/myocardial interface.

FIG. 10 depicts in-vivo 3D GRASP image of the whole heart with temporal footprint of 10 heartbeats: Typical short axis images reconstructed with and without CS are presented. Significantly improved signal-to-noise ratio (SNR) and image quality is achieved with the proposed temporal regularization.

FIG. 11 depicts an exemplary time-efficient 3D data-acquisition sequence.

FIG. 12 depicts an exemplary integrated computational framework for the highly time-resolved 3D reconstruction with motion estimation and correction, coil sensitivity estimation and CS.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. R. W. Brown et al., Magnetic Resonance Imaging: Physical Principles and Sequence Design, 2^{nd} ed., WILEY (June 2014), Braunwald's Heart Disease: A Textbook of Cardiovascular Medicine, 11^{th} ed., Elsevier (February 2018), and Allen et al., Remington: The Science and Practice of Pharmacy 22nd ed., Pharmaceutical Press (Sep. 15, 2012) provide one skilled in the art with a general guide to many of the terms in the application.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The terms “treat,” “treatment,” “treating,” or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented. Non-limiting examples of treatments or therapeutic treatments include at least one selected from pharmacological therapies, biological therapies, interventional surgical treatments, and combinations thereof. Non-limiting examples of therapeutic treatments include any one or more of coronary revascularization through stenting, coronary bypass grafting, or medical therapy, or combinations thereof. Non-limiting examples of medical therapies include statins, LDL lowering, beta blockers, ACE inhibitors, aspirin, etc. or combinations thereof.

The term “preventative treatment” means maintaining or improving a healthy state or non-diseased state of a healthy subject or subject that does not have a disease. The term “preventative treatment” also means to prevent or to slow the appearance of symptoms associated with a condition, disease, or disorder. The term “preventative treatment” also means to prevent or slow a subject from obtaining a condition, disease, or disorder.

“Diseases”, “disorder,” “conditions” and “disease conditions,” as used herein may include, but are in no way limited to any form of cardiovascular conditions, diseases or disorders. Cardiovascular diseases are a class of diseases that involve the heart or blood vessels. Non-limiting examples of cardiovascular disease include: coronary artery disease, coronary heart disease, ischemic heart disease (IHD), cardiomyopathy, stroke, hypertensive heart disease, heart failure, pulmonary heart disease, ischemic syndrome, coronary microvascular disease, cardiac dysrhythmias, rheumatic heart disease (RHD), aortic aneurysms, cardiomyopathy, atrial fibrillation, congenital heart disease, endocarditis, inflammatory heart disease, endocarditis, inflammatory cardiomegaly, myocarditis, valvular heart disease, cerebrovascular disease, and peripheral artery disease (PAD). In one embodiment of the methods, a cardiovascular disease or condition in a subject is stenosis. In another embodiment of the methods, a cardiovascular disease is ischemia. In yet another embodiment, a cardiovascular disease in the methods is perfusion defects.

A “healthy subject” or “normal subject” is a subject that does not have a disease or disorder. In some embodiments, a healthy subject, normal subject, or a control subject is a subject that does not have a cardiovascular disease.

The term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. For example, it is considered as “administering” that a subject consumes a composition as disclosed herein. In some embodiments, administering does not involve the use of a needle. In some embodiments, administering is needle-free. In some embodiments, administering is via inhalation.

“Diagnostic” refers to identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a diagnostic method may not provide a definitive diagnosis of a condition, a disease, or a disorder, it suffices if the method provides a positive indication that aids in diagnosis.

By “at risk of” is intended to mean at increased risk of, compared to a normal subject, or compared to a control group, e.g. a patient population. Thus a subject carrying a particular marker may have an increased risk for a specific condition, disease or disorder, and be identified as needing further testing. “Increased risk” or “elevated risk” mean any statistically significant increase in the probability, e.g., that the subject has the disorder. In some embodiments the risk is increased by at least 10% over the control group with which the comparison is being made. In some embodiments, the risk is increased by at least 20% over the control group with which the comparison is being made. In some embodiments, the risk is increased by at least 50% over the control group with which the comparison is being made.

The terms “detection”, “detecting” and the like, may be used in the context of detecting a condition, detecting a disease or a disorder (e.g. when positive assay results are obtained).

The term “diagnosis,” or “dx,” refers to the identification of the nature and cause of a certain phenomenon. As used herein, a diagnosis typically refers to a medical diagnosis, which is the process of determining which disease or condition explains a symptoms and signs. A diagnostic procedure, often a diagnostic test or assay, can be used to provide a diagnosis.

The term “prognosis,” or “px,” as used herein refers to predicting the likely outcome of a current standing. For example, a prognosis can include the expected duration and course of a disease or disorder, such as progressive decline or expected recovery.

A “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In some embodiments, the subject is a human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject can be one who has been previously diagnosed with or identified as suffering from or having a condition in need of treatment (e.g., a cardiovascular disease) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition or one or more complications related to the condition or a subject who does not exhibit risk factors. A “subject in need” of treatment for a particular condition can be a subject suspected of having that condition, diagnosed as having that condition, already treated or being treated for that condition, not treated for that condition, or at risk of developing that condition.

The term “statistically significant” or “significantly” refers to statistical evidence that there is a difference. It is defined as the probability of making a decision to reject the null hypothesis when the null hypothesis is actually true. The decision is often made using the p-value. In some aspects, p<0.05 indicates a statistically significant difference.

A “cardiovascular system” or “circulatory system” includes the heart and a closed system of vessels, such as arteries, veins and capillaries.

“Remote territory” means normal myocardial territory that is not affected by cardiovascular disease.

“Affected territory” means abnormal myocardial territory that is affected by cardiovascular disease.

“Rest” means before administration of a stress agent, in between administrations of a stress agent, or when the stress agent is not being administered.

“Reference image” means an image obtained before administration of a stress agent, when the stress agent is not being administered, and/or in between administrations of a stress agent to the subject.

“Stress” means after the start of administration of a stress agent and before the end of the administration of the stress agent, during the period when the stress agent is being administered, or when the cardiovascular system of the subject is functioning differently as a result of responding to an administered stress agent.

“Stimulated image” or “stress image” means an image obtained after the start of administration of a stress agent and before the end of the administration of the stress agent, or an image obtained during the period when the stress agent is being administered to the subject.

As used herein, “ms” means milliseconds; “min” or “mins” means minute or minutes; and “(ml/min/g)” means (milliliter/minute/gram).

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the numbers expressing quantities of ingredients, properties such as concentration, reaction conditions, time, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

A CMR-based approach (cfMRI denoting cardiac functional MRI) is presently described that allows for reliable detection of myocardial oxygenation by integrating repeated carbon dioxide stimulation to a patient with fast, free-breathing, high-resolution whole-heart BOLD CMR within a statistical, computational framework for analyzing BOLD CMR signals, It does this by (a) advancing a natural molecule (carbon dioxide), for repeat interrogation of the functional capacity of the heart's blood vessels; (b) developing a fast MRI approach suitable for clinical adoption, to efficiently gather time-resolved oxygenation-sensitive signals throughout the heart, without being limited by confounders such as cardiac/respiratory motion and heart-rate changes; and (c) integrating the multiple whole-heart images within a computational framework, to statistically reduce noise and arrive at confidence maps of alterations in myocardial oxygenation. At a minimum, cfMRI allows for evaluation of IHD for those currently contraindicated for current state-of-the-art imaging: it does not require ionizing radiation, contrast agents, needles (venous cannulations) or exercise. It may even prove beneficial for general application, as cost, side effects, and risk of adverse events may be less. cfMRI permits monitoring of myocardial oxygenation, which opens the potential to observe previously unattainable image data to enhance understanding of cardiac physiology and pathophysiology. This process can also be used to noninvasively identify those at risk for IHD (e.g., where impaired oxygenation may not be accompanied by detectable abnormalities in blood flow) and a diverse spectrum of heart diseases related to myocardial ischemia.

Also presently described is a method to accurately detect healthy and myocardium affected by coronary narrowing without contrast agents or ionizing radiation when (a) the heart is repeatedly stimulated with a physiologically tolerable and prospectively targeted increase in arterial CO₂ (PaCO₂); (b) the heart is rapidly imaged with a time-efficient, confounder-corrected whole-heart, free-breathing BOLD-CMR approach; and (c) the resultant BOLD-CMR images are integrated (registered and segmented) and analyzed to arrive at statistical parametric maps. We refer to this approach as cfMRI, in which CO₂ is used as a vasodilator for repeated coronary stimulation to reproducibly stimulate coronary vessels in a time-dependent manner, under monitoring and control based on breath-by-breath feedback, for a spatial coverage by whole-heart 3D BOLD-MRI scanning that is motion-insensitive, so patient can free breath, and with a fast imaging speed, e.g., each set of MR data can be obtained under 5 minutes, 4.5 minutes, 4 minutes, 3.5 minutes or less. Furthermore, computational framework is utilized including registration of multiple whole-heart images to remove motion-associated noise, reduction in noise by statistical analysis, and display in a 3D confidence maps. To statistically uncover the underlying BOLD-CMR signals in the heart, rapidly acquired images registered across multiple stimulations, as well as whole-heart BOLD-CMR images, are performed. We demonstrate the capabilities of cfMRI in a large, clinically relevant animal model with and without coronary stenosis, and conceive operating cfMRI in human subjects/patients with fast imaging to reduce hypercapnia duration without compromising accuracy in evaluating IHD, for intended diagnosis, prognosis, intervention and/or treatment.

### MRI System and Components

Various embodiments provide that an MRI system configured for a cardiac stress testing as describes includes (1) a workstation, (2) a gradient system, (3) an RF system, and (4) a gas controlling/delivery system.

In some embodiments, a workstation includes a processor, a display and a keyboard, wherein the workstation provides the operator interface that enables scan prescriptions to be entered into the MRI system. The processor can be a commercially available programmable machine which runs a commercially available operating system. Typically, the workstation is coupled to, and can communicate with each of, four servers: a pulse sequence server; a data acquisition server; a data processing server, and a data store server.

A gradient system in some embodiments includes a magnet assembly which includes a polarizing magnet, a whole-body RF coil, and a gradient coil assembly. The gradient coil assembly under excitation can produce magnetic field gradients Gx, Gy and Gz used for position encoding MR signals. The pulse sequence server functions in response to instructions downloaded from the workstation to operate a gradient system and an RF system. Gradient waveforms necessary to perform a prescribed scan are produced and applied to the gradient system that excites gradient coils in an assembly to produce the magnetic field gradients.

An RF system includes an RF transmitter for producing a wide variety of RF pulses used in MR pulse sequences, and one or more RF receiver channels. The RF transmitter is responsive to the scan prescription and direction from the pulse sequence server to produce RF pulses of the desired frequency, phase and pulse amplitude waveform. The generated RF pulses may be applied to the whole body RF coil or to one or more local coils or coil arrays. RF excitation waveforms are applied to the RF coil by the RF system to perform a prescribed magnetic resonance pulse sequence. Responsive MR signals detected by the RF coil or a separate local coil are received by the RF system, amplified, demodulated, filtered and digitized under direction of commands produced by the pulse sequence server. Typically, each RF receiver channel includes an RF amplifier that amplifies the MR signal received by the coil to which it is connected and a detector that detects and digitizes the I and Q quadrature components of the received MR signal.

The pulse sequence server also optionally receives patient data from a physiological acquisition controller. The controller receives signals from a number of different sensors connected to the patient, such as ECG signals from electrodes or respiratory signals from a bellows. Such signals are typically used by the pulse sequence server to synchronize, or “gate”, the performance of the scan with the subject's respiration or heartbeat.

Further embodiments provide the pulse sequence server also connects to a scan room interface circuit that receives signals from various sensors associated with the condition of the patient and the magnet system. Through the scan room interface circuit, a patient positioning system receives commands to move the patient to desired positions during the scan.

The digitized MR signal samples produced by the RF system are received by the data acquisition server. The data acquisition server operates in response to instructions downloaded from the workstation to receive the real-time MR data and provide buffer storage such that no data is lost by data overrun. In some scans the data acquisition server does little more than pass the acquired MR data to the data processor server. However, in scans that require information derived from acquired MR data to control the further performance of the scan, the data acquisition server is programmed to produce such information and convey it to the pulse sequence server. For example, during pre-scans MR data is acquired and used to calibrate the pulse sequence performed by the pulse sequence server. Also, navigator signals may be acquired during a scan and used to adjust RF or gradient system operating parameters or to control the view order in which k-space is sampled. And, the data acquisition server may be employed to process MR signals used to detect the arrival of contrast agent in an MRA scan. In all these examples the data acquisition server acquires MR data and processes it in real-time to produce information that is used to control the scan.

The data processing server receives MR data from the data acquisition server and processes it in accordance with instructions downloaded from the workstation. Such processing may include, for example: Fourier transformation of raw k-space MR data to produce two or three-dimensional images; the application of filters to a reconstructed image; the performance of a back projection image reconstruction of acquired MR data; the calculation of functional MR images; the calculation of motion or flow images; registration of images, etc.

Images reconstructed by the data processing server are conveyed back to the workstation where they are stored. Real-time images are stored in a data base memory cache (not shown) from which they may be output to operator display or a display that is located near the magnet assembly for use by attending physicians. Batch mode images or selected real time images are stored in a host database on disc storage. When such images have been reconstructed and transferred to storage, the data processing server notifies the data store server on the workstation. The workstation may be used by an operator to archive the images, produce films, or send the images via a network to other facilities.

A gas controlling/delivery system generally includes a gas delivery apparatus operatively connected to a processor and a gas reservoir, and a flow sensor. The gas delivery apparatus includes a breathing circuit having at least one gas conduit leading to a subject/patient's airway interface. The flow sensor is positioned to monitor in real time the rate of inspiration of a component gas. The processor can execute an algorithm to determine a desired gas composition or stress agent to be delivered through the gas conduit to a subject, monitor a cumulative volume and/or instant volume of gas, and readjust the delivery volume based on monitored volumes or a predetermined threshold. An exemplary gas controlling/delivery system is the RESPIRACT™ platform from Thornhill Research. Detail on RESPIRACT platform is described in United States Patent Application Publication Nos. 2014-0311491 and 2015-0034085, which are herein incorporated by reference in their entirety.

### Process of Using an MRI System to Perform Cardiac Stress Testing

Methods are provided for operating an MRI system and/or performing a cardiac stress test in a subject, and the methods include directing the MRI system to scan the cardiovascular system of the subject at at least two different states, e.g., including a rest (e.g., when no stress agent is administered to the subject) or a reference state, and a stress state where one or different amounts of the stress agent is administered to the subject (different amounts counted as different states), such that multiple MRI acquisitions are performed at the at least two states. In some embodiments, MRI acquisitions are performed at two different states; some embodiments, MRI acquisitions are performed at three different states; and some embodiments, MRI acquisitions are performed at four, five or more different states; wherein at least one of the state is when the stress agent is administered in an amount that causes increased blood velocity/flow rate such that MRI acquisition discerns the difference from a rest state. When MRI acquisitions are performed at only two states, typically multiple acquisitions are needed at each of the two states, such that the number of acquisitions can lead to a statistical power of 0.8 or greater for comparison of images between the two states to discern any statistically significant difference due to the cardiovascular system's response to the stress agent. When MRI acquisitions are performed at three or more states, i.e., including at least two different levels of stress agents administered to the subject, a generalized linear model to make a regression of the MRI voxels/pixels characteristics of blood oxygen/volume/flow over the amount of administered stress agents can discern if the stress agent has an effect on the blood oxygen/volume/flow, wherein no effect by the stress agent indicates the cardiovascular system has impaired function when the statistical power of the regression is typically 0.8 or greater, given that at least one amount of the stress agent is high enough to cause increased blood velocity or flow rates in a normal subject without cardiovascular disease or in a healthy/normal portion of the cardiovascular system in the subject at test.

Some embodiments provide obtaining MR data in a repeated, successive or intermittent manner (denoted as Rᵢ, e.g., R₁, R₂, R₃, R₄, etc.) thereby obtaining a plurality of reference images while the subject is at rest (with no stress agent) or at a baseline PaCO₂ level (e.g., R₁ image, R₂ image, R₃ image, R₄ image, etc.); further directing the MRI system to scan the cardiovascular system at a stressed state (e.g., when stress agent is administered and/or maintained with the subject) to obtain MR data that can be reconstructed as a stress image of the cardiovascular system, which is performed in a repeated, successive or intermittent manner (denoted as S₁, e.g., S₁, S₂, S₃, S₄, etc.) thereby obtaining a plurality of stress images (e.g., S₁ image, S₂ image, S₃ image, S₄ image, etc.); and comparing, or directing a processor to compare, the plurality of the reference images with the plurality of the stress images in terms of MR data (e.g., image voxels or pixels).

Without being bound by a theory, one MRI image at either rest or stress state may have a high noise level, which impedes discrimination between the rest state and the stress state. Increasing the number of obtained MRI images, in various embodiments, reduces noise in MR data and increases statistical power when combining the plurality of images or looking at them as a whole, thereby increasing specificity and accuracy in discriminating the subject's response at the rest state and the stress state.

In some embodiments, an order of the repetitions, successions or intermittence is R₁, then S₁, then R₂, then S₂, then R₃, then S₃, then R₄, and followed by S₄ etc. That is, the subject is imaged at an alternating condition in pairs of a rest state followed by a stress state, e.g., for at least 2 pairs, at least 3 pairs, at least 4 pairs, at least 5 pairs, or until the statistical power in comparing between the plurality of the reference images and the plurality of the reference images is at least 0.8.

In some embodiments, an order of the repetitions, successions or intermittence is S₁, then R₁, then S₂, then R₂, then S₃, then R₃, then S₄, and followed by R₄ etc. That is, the subject is imaged at an alternating condition in pairs of a stress state followed by a rest state, e.g., for at least 2 pairs, at least 3 pairs, at least 4 pairs, at least 5 pairs, or until the statistical power in comparing between the plurality of the reference images and the plurality of the reference images is at least 0.8.

In some embodiments, an order of the repetitions, successions or intermittence is R₁, R₂, R₃, R₄ etc., followed by S₁, S₂, S₃, S₄ etc. That is, the subject is imaged at a rest state successively to obtain a plurality of reference images, and followed by being imaged at a stress state successively to obtain a plurality of stress images, which may optionally be further followed by being imaged at the rest state to obtain another set of reference images and being imaged at the stress state to obtain another set of stress images.

In some embodiments, an order of the repetitions, successions or intermittence is S₁, S₂, S₃, S₄ etc., followed by R₁, R₂, R₃, R₄ etc. That is, the subject is imaged at a stress state successively to obtain a plurality of stress images, and followed by being imaged at a rest state successively to obtain a plurality of reference images, which may optionally be further followed by being imaged at the stress state to obtain another set of stress images and being imaged at the rest state to obtain another set of reference images.

In some embodiments, an order of the repetitions, successions or intermittence is R₁, R₂, S₁, S₂, R₃, S₃, R₄, S₄, or another one wherein the subject is imaged at a rest state for a number of times that are interspersed with being imaged at a stress state for a number of times.

In further embodiments, the subject is imaged at a randomized order of a number of rest states and a number of stress states.

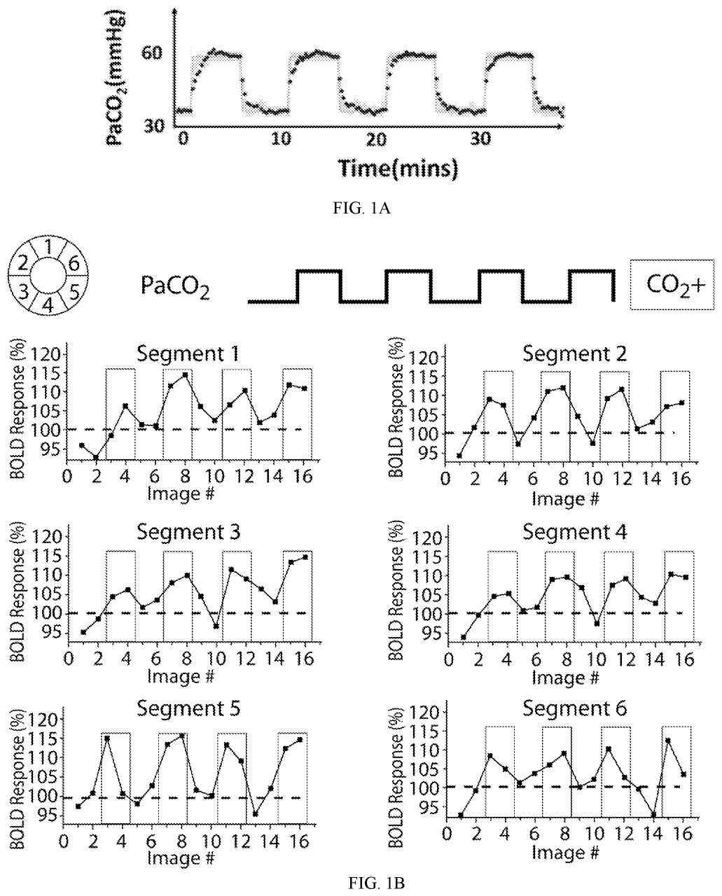

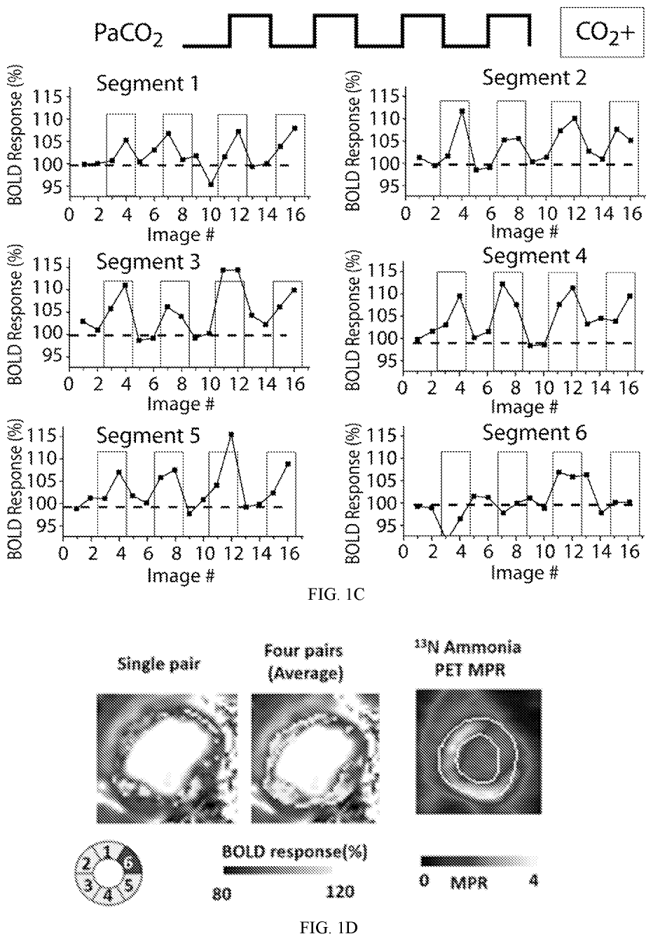

The strength of the stress states can be adjusted by changing the amount (e.g., per unit time and per unit volume) of the stress agent administered to the subject. Increasing the concentration or flux of the stress agent administered to the subject typically increases the strength of a stress state. In one embodiment, a fixed amount of a stress agent is administered to the subject in each stress state, such that the strength of the stress states (e.g., the amount of the stress agent in the subject) over a number of the stress states is a square wave pattern, e.g., as seen in FIG. 1A. In another embodiment, a defined varying amount of a stress agent is administered to the subject in each stress state, such that the strength of the stress states (e.g., the amount of the stress agent in the subject) over a number of the stress states is a saw-tooth pattern, e.g., with steadily increased stress strength in each stress state with interspersed rest states. In another embodiment, a defined varying amount of a stress agent is administered to the subject in one or more stress states, such that the strength of the stress states (e.g., the amount of the stress agent in the subject) over a number of the stress states is an oscillatory pattern. In yet another embodiment, in one or more stress states a stress agent is administered in a combination of a defined varying amount and a fixed amount.

In some embodiments, the plurality of reference images has a number that is, or the number of sets of MR data obtained when the subject is at a rest state is, the same as the number of the plurality of stress images, or the number of sets of MR data obtained when the subject is at a stress state. In other embodiments, the plurality of reference images has a number that is, or the number of sets of MR data obtained when the subject is at a rest state is, not the same as the number of the plurality of stress images or the number of sets of MR data obtained when the subject is at a stress state. Further embodiments provide that the plurality of reference images and the plurality of stress images are large enough in numbers such that a statistical power is at least 0.8.

Hence some embodiments of the methods include directing the MRI system to obtain at least 4 sets of MR data in order to acquire at least 4 reference images of the cardiovascular system, and directing the MRI system to obtain at least another 4 sets of MR data to acquire at least 4 stress images of the cardiovascular system.

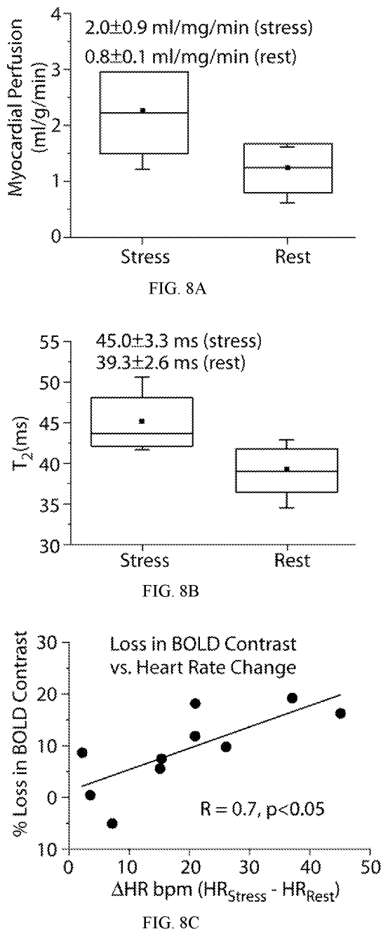

Further embodiments provide administering the stress agent in a stepwise manner of increments of 5 mmHg, 10 mmHg, 15 mmHg, 20 mmHg, 25 mmHg or another amount, such that multiple MRI acquisitions can be made at different levels of administered stress agent, e.g., FIG. 8C, and when the acquisitions are sufficient in number (e.g., leading to a statistical power of at least 0.8), a statistically significant difference or effect due to the stress agent made on the MRI image voxels/pixels characteristics of blood oxygenation/volume/flow is indicative that the blood vessels are normal, whereas a lack of a statistically significant difference or effect due to the stress agent made on the MRI image voxels/pixels is indicative that the blood vessels have stenosis or are indicative of ischemia.

Yet other embodiments provide administering the stress agent in a sinusoidal manner, or another varying manner, wherein multiple MRI acquisitions can be made at different stress levels (some embodiments including zero stress) for the statistical analysis.

Additional embodiments of the methods further include performing a pre-scan before directing the MRI system to acquire a first set of stress MR data, in order to test an amount of a stress agent and/or a sequence of MRI so as to adjust the strength (e.g., concentration, amount and/or duration) of the stress agent and/or the sequence of MRI, if needed, during MR scanning. Exemplary pre-scan procedures include administering an amount of a stress agent to the subject, monitoring the subject's response to the administered stress agent, and/or directing the MRI system to perform a sequence to acquire MR data while the stress agent is administered, wherein if the subject's response to the stress agent in the pre-scan is intolerable or unsafe, a lower dosage (e.g., in concentration and/or duration) than that in the pre-scan is utilized to conduct a repeat of the pre-scan or in obtaining MR data under the stress state; and if the subject's response in the pre-scan step is tolerable or safe, and the MR data reflects the difference due to the stress agent, a same dosage is utilized in obtaining MR data under the stress state, or a higher dosage (e.g., in concentration and/or duration) is utilized in another pre-scan step to determine if the higher dosage can be utilized in acquiring stress MR data in a safe and/or tolerable manner for the subject.

Further embodiments provide a fast, whole-heart (e.g., human heart; canine heart) 3D imaging by the MRI system, in which each MR scan while the subject is under stimulation with the stress agent to obtain MR data, for eventually a stress MR image reconstruction, i.e., each duration of administration of the stress agent, is less than 6 minutes, less than 5 minutes, less than 4 minutes, less than 3 minutes, less than 2 minutes, or less than 1 minutes. In one aspect, a whole-heart 3D imaging by the MRI system include compressive sensing sampling, such that each MR scan to obtain MR data (for eventual reconstruction of an MR image of the whole heart) is less than 4 minutes; and a total number of 4 times of reference MR scans (to obtain 4 reference images in total of the whole-heart) and 4 times of stress MR scans (to obtain 4 stress images in total of the whole-heart) provide effective amounts of voxels for comparing between the reference images and the stress images, to allow for a statistical power of greater than 0.8, and/or a greater than 95%, 96%, 97%, 98%, 99%, 94%, 93%, 92%, 91% or 90% accuracy in indicating impaired cardiovascular function compared to results from ¹³N PET scan.

Other embodiments of the methods further include calculating the statistical power for comparing between a plurality of reference images and a plurality of stress images, and if the statistical power is less than 0.8, repeating the steps of directing the MRI system to acquire one more reference image, administering the stress agent while directing the MRI system to acquire one more stress image, and followed by recalculating the statistical power; and if the statistical power is 0.8 or greater, the method can proceed to comparing the plurality of reference images with the plurality of stress images in terms of image voxel or pixel characteristic of blood oxygenation, blood volume and/or blood flow. Hence, some embodiments of the methods include multiple measurements (MR scanning and image processing) over a period of time to acquire increasing response (e.g., increased overall signal to noise ratio to result in true positive signal—functional normal blood vessels responding to the stress agent) so as to arrive at a statistical power of response of 0.8.

Comparing Step

When comparing the plurality of reference images with the plurality of stress images, which includes comparing a certain segment or region of the cardiovascular system of the subject in the plurality of the reference images versus that segment or region in the plurality of the stress images, a statistical analysis is generally applied. Exemplary statistical analysis techniques suitable for the methods disclosed herein include, but are not limited to, ANOVA, T-test, paired T-test, chi-square test, ANCOVA, factor analysis, cluster analysis, with use of Fisher hypothesis, Neymanian hypothesis, or Bayes factors; or comparing between the mean or median values.

Various embodiments provide that the comparison is in terms of MR data, or the image pixels or voxels, representative of a property such as blood oxygenation, blood volume, and/or blood flow of the cardiovascular system. Aspects of the disclosed methods provide that the stress agent is administered in an amount effective to induce changes in at least some regions of the cardiovascular system of the subject. Further embodiments provide that the stress agent is administered in an amount that is biologically safe to the subject and induces reversible changes in regions of the cardiovascular system, wherein the changes can revert or disappear after the stress agent is removed from the subject. Hence, a region in the cardiovascular system unable to respond to the effective amount of the stress agent, indicated by showing no changes/difference in the images from a rest state to a stress state, can be inferred as having impaired function (blood oxygenation, blood volume and/or blood flow).

Generally, a difference between the plurality of the reference images and the stress images is indicative that the cardiovascular system (or a region thereof wherein the difference occurs) is able to respond to the stress agent in a way that exhibits changes from the rest state; and a lack of a difference between the plurality of the reference images and the stress images indicates that the cardiovascular system (or a region thereof wherein there is the lack of a difference) is unable to respond to the stress agent, thereby exhibiting no change or difference compared to the rest state.

Further, using a generalized linear model, if the stress agent has an effect on the Mill voxels/pixels, i.e., p<0.05 when analyzing the MRI contrasts acquired at different levels of the stress agent, then the cardiovascular system is normal, and if the stress agent has no effect on the MRI voxels/pixels, i.e., p>0.05, then the cardiovascular system is impaired, e.g., ischemic and/or stenosis.

Hence in some embodiments, a null hypothesis in a statistical analysis is there is no difference in the response between the rest state and the stress state, wherein an absence in the statistical analysis of a significant difference indicates that the null hypothesis is true or cannot be rejected, and that the subject has impaired cardiovascular function in the region that shows no difference in the response between the rest state and the stress state, and wherein a presence of a significant difference indicates that the null hypothesis is rejected, and that the subject's cardiovascular function in the region that shows significant different in the response between the rest state and the stress state is normal. These methods can be used in detecting presence, progression and/or outcome of ischemia in a subject in need of diagnosis or prognosis of a cardiovascular disease and/or evaluation of recovery from a cardiovascular disease.

In other embodiments, a plurality of reference, or stress, images are obtained from a subject in need of diagnosis or prognosis of a cardiovascular disease, and a plurality of reference, or stress, images, respectively are obtained from a control subject not having a cardiovascular disease or having been examined to be free of a cardiovascular disease; and a null hypothesis is there is no difference between the plurality of reference, or stress, images of the subject in need of diagnosis or prognosis and the plurality of reference, or stress, images, respectively, of the control subject. Then an absence of a significant difference in a region when combining each subject's plurality of images indicates null hypothesis is true and the subject in need of diagnosis or prognosis does not have the cardiovascular disease in that region; and a presence of significant difference in a region when combining each subject's plurality of images indicates null hypothesis is rejected and the subject in need of diagnosis or prognosis has a diagnosis or prognosis of the cardiovascular disease in that region.

MRI Sequences (RF Pulses and Readout)

In various methods, operating an MRI system includes directing an MRI system to perform a sequence, also roughly called scanning, in order to obtain MR data and reconstruct the data to acquire MR images. Typically in each acquisition (i.e., collection of a series of frames of data), an RF excitation produces new transverse magnetization, which is then sampled along a trajectory in k-space.

Various embodiments of the disclosed methods utilize an MRI sequence with the following features: a. a sequence that is sensitive to one or more contrasts including blood oxygenation, blood volume, and blood flow; b. a sequence that allows for acquisition without breath holds by the subject, e.g., including pulse(s) and/or MR data processing technique(s) that allow for motion correction; c. a sequence that allows for reduction or elimination of imaging confounders such as heart rate dependency and magnetic field inhomogeneity (e.g., B₀, B₁ artifacts); d. a sequence that can cover partial or whole myocardium, e.g., a three-dimensional imaging. One embodiment provides a time-efficient, confounder-corrected, whole-heart, free-breathing, heart rate variation independent, and 3D (three-dimensional) or 2D (two-dimensional), and also in a motion-corrected manner. Other embodiments provide exemplary pulses or techniques for motion correction, suitable for use in the methods, including performing the motion control during the acquisition (e.g., a “navigator” technique, PACE (prospective acquisition correction), “periodically rotated overlapping parallel lines with enhanced reconstruction” technique which collects data in concentric rectangular strips rotated about the k-space origin, an octant or cloverleaf navigator approach based on improved k-space trajectory and associated mapping procedure to allow rapid, inline correction), as well as post-processing strategy (e.g., registration techniques, motion estimation), and those understood by one skilled in the art.

In some embodiments, motion-correction pulses include but are not limited to navigator gated sequences, rigid/non-rigid motion registered images, and motion resolved images using ‘advance reconstruction algorithms’. ‘Advance reconstruction algorithms’ include but are not limited to parallel imaging, compressed sensing, low-rank tensor formulation, machine learning, deep learning.

Further embodiments provide exemplary pulses or techniques for confounder reduction/elimination, suitable for use in the methods, as described below and those understood by one skilled in the art. In some embodiments, confounder reduction Mill sequences include but are not limited to (1) saturation recovery pulses to compensate for heart-rate dependency, (2) quantitative mapping to compensate for coil sensitivity bias, (3) adiabatic pluses to reduce B₁ dependency, and (4) gradient echo readout to minimize sensitivity to B₀ inhomogeneity.

Further embodiments provide that the performed sequence is sensitive to blood oxygenation, e.g., by using T2 map, T2* map, T2 or T2* map, T2 or T2*-weighted, corrected T2 or T2*-weighted images in an Mill system (e.g., BOLD-MRI system). Yet some embodiments provide that the performed sequence is sensitive to blood volume, e.g., by using T1 map or T1-weighted map in an Mill system. Another embodiment provide that the performed sequence is sensitive to blood perfusion, e.g., by using arterial spin labeling.

For example, MR sequences sensitive to blood oxygenation are based on changes in magnetic susceptibility of hemoglobin as it releases oxygen, which induces perturbations of the magnetic field inside and outside the vessels, thereby decreasing the T2* relaxation time in an imaging voxel. T2* is related to the total amount of deoxyhemoglobin in the voxel and, by extension, the blood oxygen saturation and partial pressure of oxygen in and around blood vessels. Various embodiments of the methods include operating a BOLD-MRI system to acquire T2* mapping data, T2 mapping data, T2 or T2*-weighted data, or corrected T2 or T2*-weighted data, or related data, to acquire MR images indicative of blood oxygenation in an imaged tissue without the use of a contrast agent, i.e., contrast agent-free.

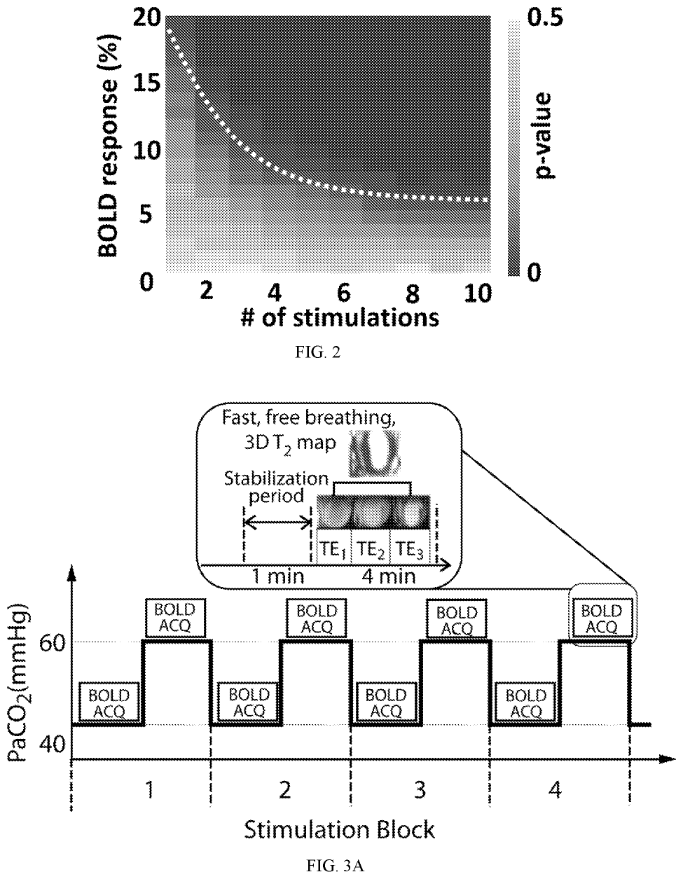

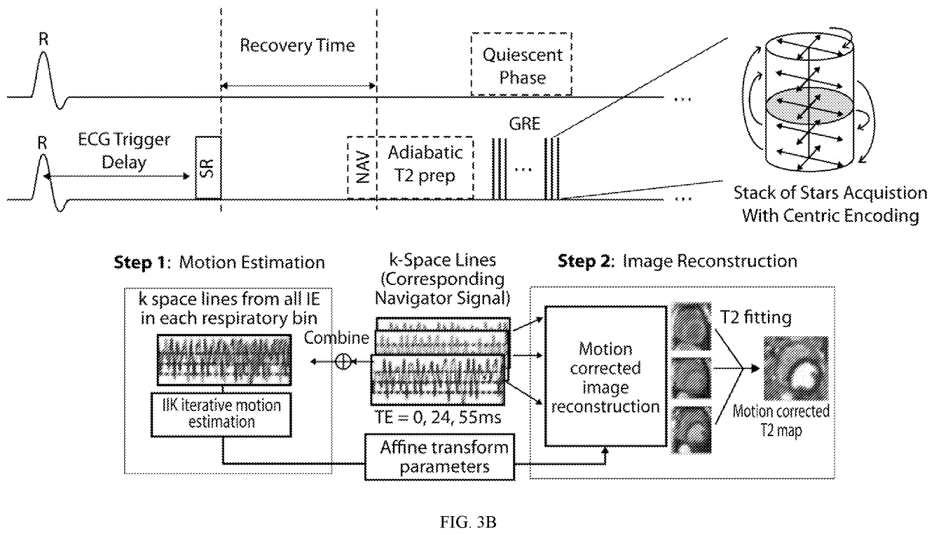

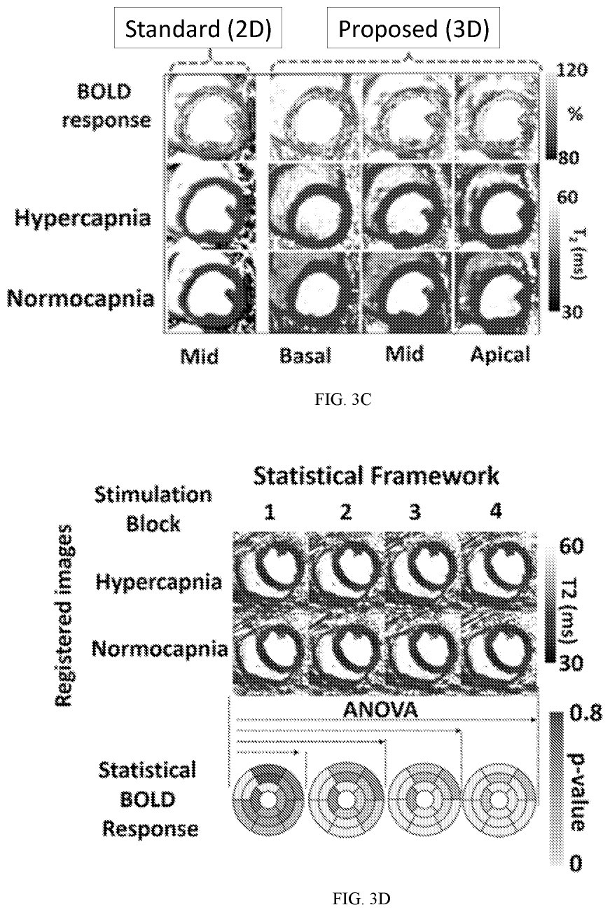

Some embodiments of the methods include directing the MRI system to perform a sequence in obtaining a set of MR data, wherein in a heartbeat (with the time before the next heartbeat) after an R-wave in ECG and an ECG trigger delay, a few “preparation” pulses are performed: a saturation recovery (SR) pulse is performed, followed by a recovery time, a navigator (NAV) pulse is performed, and adiabatic T2 preparation pulse is performed, and for “readout” a gradient echo (GRE) pulse is performed. This is exemplified in FIG. 3B. The SR preparation eliminates signal dependence on heart rate between segmented readouts. The NAV preparation allows for monitoring of the respiratory motion during acquisition. The adiabatic preparation with spoiled GRE readout minimizes B₀ and B₁ artifacts, which can otherwise be prominent at 3T and confound BOLD signal readouts. In some aspects, MR data is acquired at every heartbeat. In other aspects, MR data is acquired at every other heartbeat.

Further embodiments of the methods include directing the MRI system to perform a sequence in obtaining a set of MR data, wherein after an R-wave in electrocardiogram (ECG), a few “preparation” pulses are performed: a SR pulse is performed, followed by a fixed recovery time, and an adiabatic T2 pulse is performed, and for “readout” a GRE pulse is performed. This is exemplified in FIG. 11.

Other embodiments of a suitable sequence include the use of a saturation prepulse to reset magnetization every heartbeat, so as to afford insensitivity to heart rate variability. In various aspects, a SAT pulse is applied at the start of every heartbeat to reduce variations in signal intensities. Various aspects provide that in order to prevent changing heart rates from affecting the degree of T1-weighting in the images, the T_{SAT} (SAT delay, duration between SAT and T2 Prep) is kept constant even when the T_{trigger} (trigger delay, imaging delay after ECG R wave is detected) is allowed to change to maintain imaging during diastole in the presence of heart rate variability.

Various embodiments of the methods utilize one or more data acquisition strategies. An exemplary data acquisition strategy suitable for the disclosed methods is stack-of-stars sampling, golden-ratio rotated stack-of-stars sampling, radial sampling, spiral sampling, compressive sampling, optionally with centric encoding, or a combination thereof.

Further embodiments of the invention include directing Mill to perform sequences or data acquisition, processing techniques as described PCT application publication WO2019210145, which is incorporated by reference in its entirety.

Other RF pulses and/or readout techniques are understood by one skilled in the art to be suitable for use, in addition to or in replacement of some of the above-exemplified sequences, in the described methods.

A 90-degree pulse is of an energy exactly enough to rotate the magnetization by 90 degrees, so the net magnetization is rotated from the z-axis (z-axis parallel to B₀) into the xy-plane. At that point, M_{z}, the magnetization along B₀, is 0. In gradient-echo sequences, less energy can be put in to give a rotation of less than 90 degrees.

In inversion recovery (IR) sequences, a 180-degree pulse to ‘flip’ the magnetization vector into the −z direction is employed, which is twice as long (or strong) as the 90-degree pulse.

The spin echo (SE) sequence uses an additional, 180-degree pulse, to flip the transverse magnetization to generate an echo as they rephase. This minimizes T2* effects from slowly varying magnetic field inhomogeneity. It also provides improved signal characteristics but is slower to acquire. Fast spin echo (FSE) uses multiple successive 180-degree pulses to speed up the acquisition. A spin echo sequence has 3 main parts: (1) 90-degree pulse followed by free induction decay; (2) 180-degree pulse followed by rephasing; and (3) the echo that occurs at TE, when the scanner acquires the signal (referred to as readout). One would need to repeat this sequence a number of times to acquire the entire image, and the time between each repetition of the sequence is referred to as the repetition time or TR. One would need to wait this time in order to allow the longitudinal magnetization to recover to equilibrium, since it is used to generate the transverse magnetization (by the 90-degree pulse) in the next repetition.

A GRE sequence does not use a 180-degree refocusing pulse and thus retains T2* dephasing. An advantage of GRE is that one can use very short TR (since one does not need to wait for the 180 degree pulse and rephasing). The TRs are sufficiently short that there is some residual transverse magnetization left over at the end of each repetition. Successive pulses (repetitions) in the GRE sequence can create spin echoes, which will alter the image contrast. To get around that, a strong ‘spoiling’ or ‘crushing’ gradient at the end of each repetition can be employed to completely dephase the residual transverse magnetization.

Instead of spoiling the residual transverse magnetization at the end of each repetition, one can keep it and use it to add to our signal. “Steady-state” MR sequences can be performed because they keep the transverse magnetization for each repetition, eventually reaching an equilibrium or steady-state of transverse magnetization. One example of steady-state free precession (SSFP) sequences is balanced (also known as fully refocused) SSFP sequences. The balanced SSFP sequence is designed to fully compensate for all gradient dephasing from one repetition to the next. That is, for each gradient applied, a reverse gradient is applied at the end of the sequence to reverse its effects. This approach generally maximizes the obtained signal, since one reverses gradient-based dephasing. Additionally, the balanced SSFP sequence is relatively insensitive to motion and blood flow-based dephasing, since the balanced gradients have the triphasic design. The balanced SSFP sequence can be extremely fast, with a TR (RF spacing) of approximately 3.5 milliseconds, since one is using a steady-state magnetization that is in equilibrium between excitation and relaxation.

Echo planar imaging (EPI) is another, yet perhaps a fast sequence available and suitable for e.g., diffusion-weighted imaging. It essentially forms rapidly alternating gradient echoes within a spin echo sequence.

Adiabatic pulses that define an amplitude modulation and a frequency modulation are applied in a sequence of pulses to obtain a T2 weighted magnetic resonance image. Such an adiabatic T2 prep sequence typically includes a first 90° pulse, an even number of adiabatic pulses, and a second 90° pulse. Further examples of adiabatic pulses are described in U.S. Pat. No. 7,788,930, which is hereby incorporated by reference in its entirety.

Reconstruction, Registration and Segmentation

In various embodiments of the invention, MR data is used to reconstruct MRI mapping. Exemplary mapping includes T2 mapping, T1 mapping, T2* mapping, and combinations thereof. Exemplary reconstruction techniques suitable for use in the invention include applying inverse fast Fourier transform, using filtered back-projection, interpolation scheme, or others known in the art in manners such as iterative reconstruction.

Some embodiments provide the invention utilizes iterative image reconstruction for radial encodings in MRI acquisitions based on a (e.g., temporal) total variation regularization.

Further embodiments provide the invention utilizes compressed sensing to improve image reconstruction.

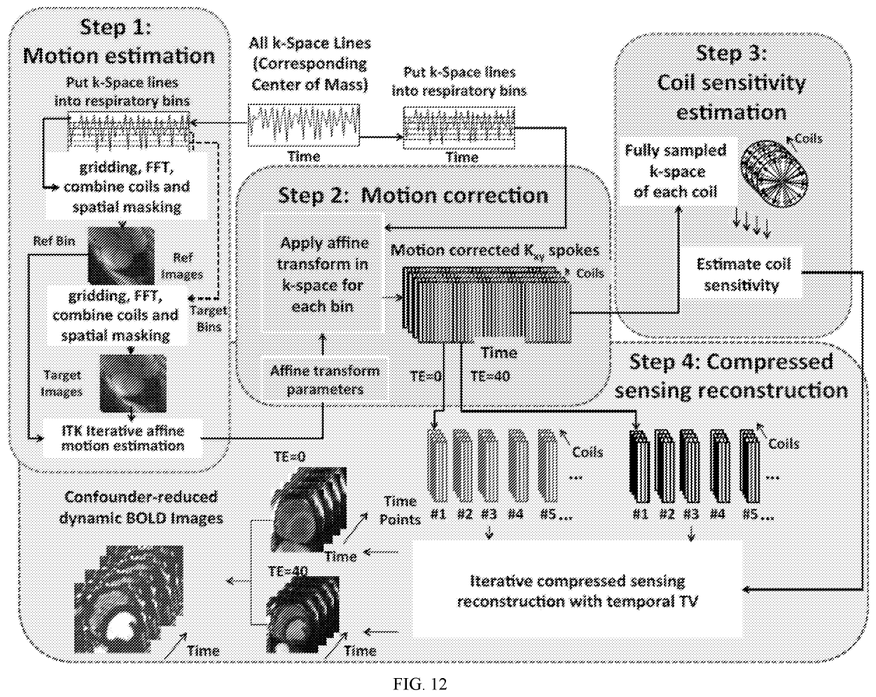

Other embodiments provide the invention includes reconstructing time-resolved, motion-compensated images by applying motion estimation, motion correction, and optionally coil sensitivity estimation, in a compressed sensing reconstruction strategy, as described in FIG. 12 and in Example 2-1.

Some embodiments of the invention include performing respiratory motion correction by sorting k-space lines into a plurality of bins (e.g., 6, 4, 5, 7, 8 etc.), each corresponding to a respiratory state, with a first bin (e.g., end-expiration) as reference and a last bin as end inspiration; registering images from other bins based on the first reference bin, wherein transformation parameters are estimated between different bins; dividing the k-space data again into the plurality of bins; and applying estimated affine transforms to corresponding lines to obtain a motion-free dataset.

Further embodiments of the invention include increasing temporal resolution by separating the motion-free dataset based on acquisition coils, wherein corresponding coil sensitivity is estimated by combining all k-space lines; splitting k-space lines into subsets based on echo times (TEs); and applying iterative radial reconstruction with temporal total-variance constrains for each plurality (e.g., 55, 56, 57, 58, 59, 60, 50, 51, 52, 53, 54) of consecutive radial lines in each subset of k-space lines. In some aspects, the invention achieves a 5-fold, 6-fold, 7-fold, 8-fold, 9-fold, or 10-fold acceleration without degrading perceptible image quality. In further aspects, dynamic image series from each TE subset is reconstructed using a constrained optimization algorithm, e.g.,

$$argmin ( {‖ F \cdot c \cdot d - m ‖}_{2}^{2} + λ \vert T \cdot d {\vert}_{1} ) ,$$

where F is non-uniform fast Fourier transform (NUFFT), c is coil sensitivity, d is the estimated time series of images, m is the measurement, γ is the weight of regularizer, and T is the temporal TV operator.

Various embodiments of the invention further include segmenting the acquired MR images/data based on structure of the cardiovascular system, e.g., to isolate myocardium, registering the segmented images/data to obtain motion-corrected MR images/dataset, and comparing the MR images/dataset of the segment in a plurality of MR scans at the rest state of the subject (e.g., when the stress agent is not administered, or the subject is in a normocapnia state) with that in a plurality of MR scans at the stress state (e.g., when the stress agent is being administered, or hypercapnia state).

Exemplary myocardial segmentation arrangements include those defined by American Heart Association (AHA), with divisions into a variable number of segments; or other user-defined arrangements. In one embodiment, the invention includes segmenting the MR imaged heart into six segments, as exemplified in Example 1 and FIGS. 1B and 1C. In another embodiment, the invention includes segmenting left ventricle into 16 segments, as exemplified in FIG. 3D, or 17 segments (further counting the center segment identifying apex). The 16-segment model by standards of AHA can be arranged as a polar plot with the apex in the center, four apical segments as a first ring, six mid-cavity segments as the second ring, and six apical segments as the outermost ring.

Further embodiments provide that segmenting the cardiovascular system includes at least a segment of endocardium and a segment of epicardium, and a lack of a statistically significant difference in the image voxels or pixels in the endocardium segment between the motion-corrected reference images and the motion-corrected stress images and a lack of a statistically significant difference in the image voxels or pixels in the epicardium segment between the motion-corrected reference images and the motion-corrected stress images is indicative of balanced myocardial ischemia in the subject.

In some embodiments, dataset is analyzed by fitting voxel-based supervised (e.g., general linear model) or unsupervised (e.g., independent component analysis) models. In one embodiment, at least 10 data points are generated for each volume of 144×144×12 voxels in either a normocapnic or a hypercapnic condition (stimulation block in FIG. 3A), and a concatenated, fully registered dataset for a study will contain 144×144×12×N voxels, where N=10K, and K is the number of normocapnic or hypercapnic blocks (e.g., K is an integer from 1 to 6).

Statistical Parametric Map (SPM)

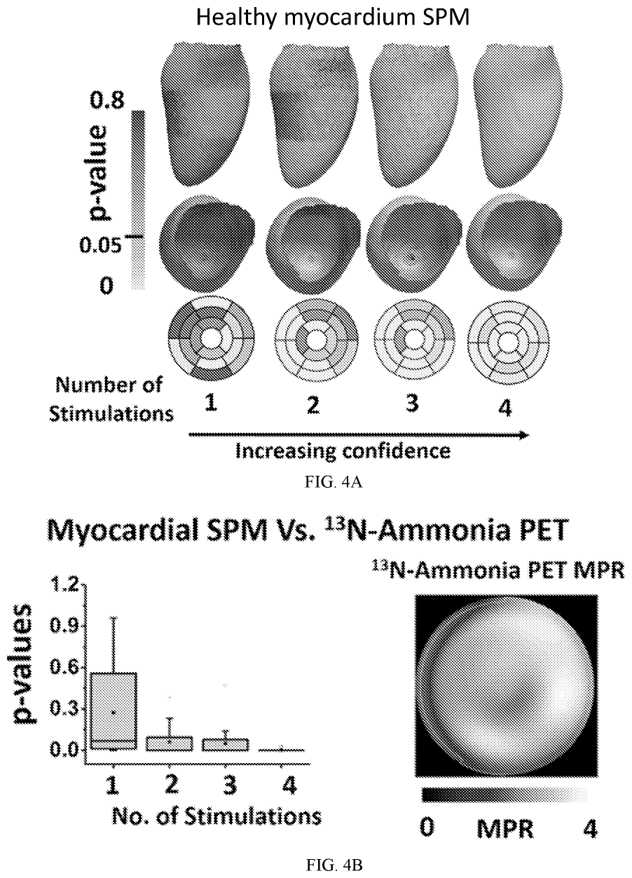

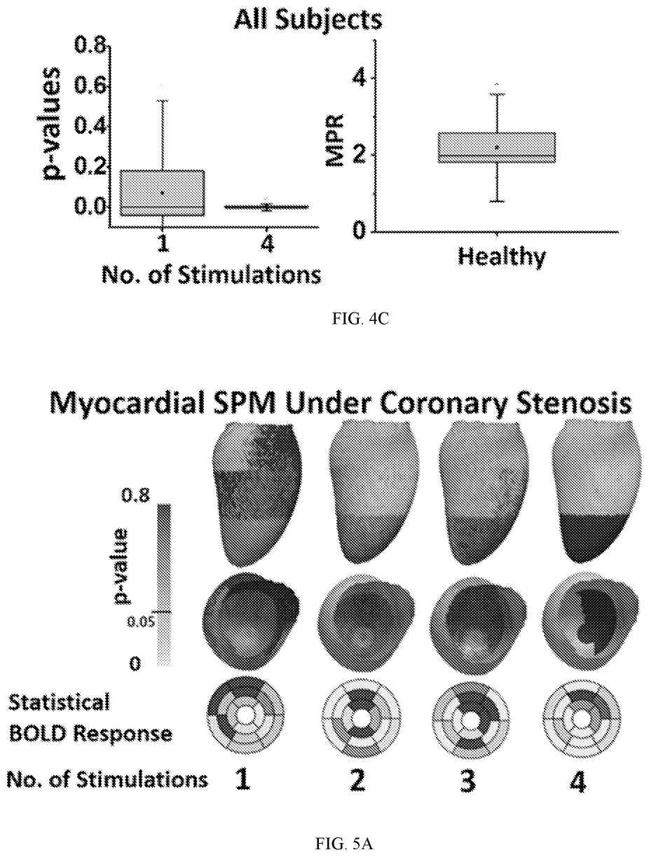

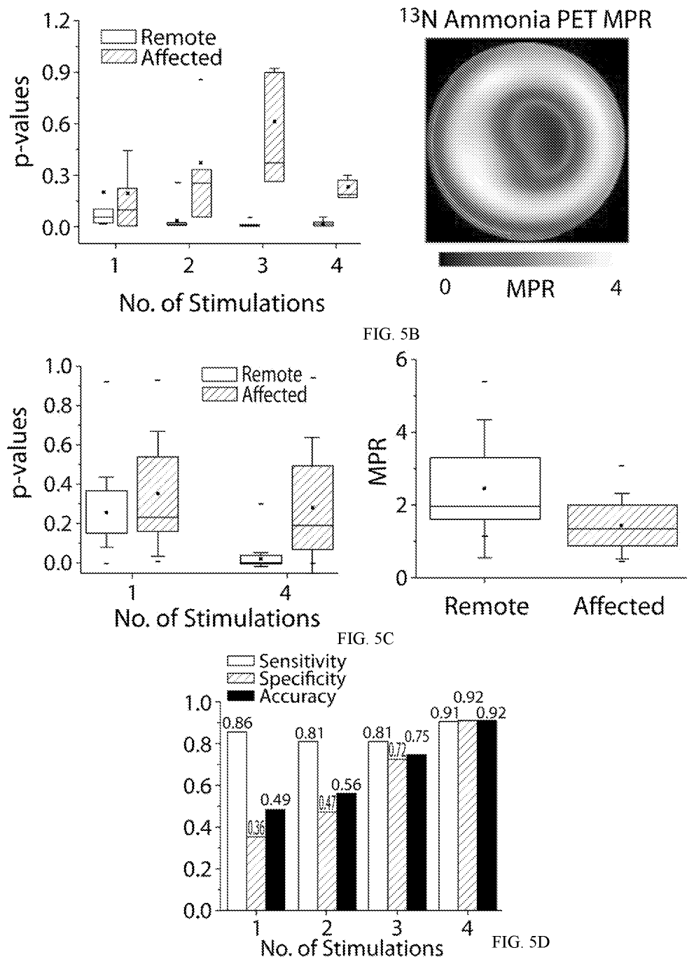

Various embodiments of the invention provide a statistical parametric map, which contains a P-statistic volume on a voxel-by-voxel basis of a cardiovascular system (e.g., myocardium), wherein the p-value of significance of each voxel in comparing the plurality of reference images and the plurality of stress images is shown in an image or field. Exemplary SPMs of myocardium are shown in FIGS. 4A and 5A.

In further embodiments, voxels with a p-value below a threshold (e.g., p<0.5) is identified as responsive to the stress agent (e.g., hyperemia in response to hypercapnia). In some aspects, relative hyperemic volume in response to a stress agent is calculated as the total responding voxels relative to the total voxels in myocardium.

Stress Agent

One or more stress agents can be applied in the methods disclosed herein. Various embodiments provide that the stress agents are used to cause increased blood velocity and flow rate in normal vessels and less (or none) of a response in stenotic vessels, which can be identified using imaging systems such as MRI. In various embodiments, the stress agent is carbon dioxide (CO₂). In various embodiments, the stress agent is an admixture comprising carbon dioxide (CO₂). In some embodiments, the admixture comprising carbon dioxide (CO₂) further comprises oxygen (O₂). In some embodiments, the admixture comprising carbon dioxide (CO₂) is carbogen. “Carbogen” as used herein is an admixture of carbon dioxide and oxygen. The amounts of carbon dioxide and oxygen in the admixture may be determined by one of skill in the art. The amounts of carbon dioxide and oxygen in the admixture may be determined by one of skill in the art. Medical grade carbogen is typically 5% CO₂ and 95% O₂. In various embodiments, carbon dioxide is used to induce hyperemia may be an admixture of ranges including but not limited to 94% O₂ and 6% CO₂, 93% O₂ and 7% CO₂, 92% O₂ and 8% CO₂, 91% O₂ and 9% CO₂, 90% O₂ and 10% CO₂, 85% O₂ and 15% CO₂, 80% O₂ and 20% CO₂, 75% O₂ and 25% CO₂, and/or 70% O₂ and 30% CO₂.

In some embodiments, the admixture comprises any one or more of carbon dioxide, oxygen and nitrogen, and optionally water vapor; carbon dioxide and oxygen; carbon dioxide and nitrogen; or carbon dioxide alone. In one embodiment, the amounts of CO₂ and O₂ administered are both altered. In some embodiments, the amount of CO₂ administered is altered to a predetermined level while the amount of O₂ administered is held constant. In some embodiments, the amounts of any one or more of CO₂, O₂ or N₂ in an admixture are changed or held constant as would be readily apparent to a person having ordinary skill in the art.

Further embodiments provide the stress agent in one or more of the methods is any form of a repeatable stressor. For example, the stress agent can be regadenoson, adenosine, dipyridamole, dobutamine, CO₂, or a combination thereof.

Various embodiments of the invention include administering to the subject an effective amount of a stress agent, wherein in some aspects, the amount is effective to induce hyperemic response in the subject; in some aspects, the amount is effective to induce hyperemic response in one or a group of subjects free of cardiovascular disease; in some further aspects, the amount is effective to result in statistical significant difference in terms of blood oxygenation, volume and/or perfusion in the MR dataset compared to that obtained when the subject is at rest.

In some embodiments, the stress agent (e.g., an admixture comprising CO₂) is administered at high doses for short durations each time (and possibly short durations when all repetitions combined). For example, administering the admixture comprising CO₂ at high doses of CO₂ for a short duration comprises administering any one or more of 40 mmHg to 45 mmHg, 45 mmHg to 50 mmHg, 50 mmHg to 55 mmHg, 55 mmHg CO₂ to 60 mm Hg CO₂, 60 mmHg CO₂ to 65 mm Hg CO₂, 65 mmHg CO₂ to 70 mm Hg CO₂, 70 mmHg CO₂ to 75 mm Hg CO₂, 75 mmHg CO₂ to 80 mm Hg CO₂, 80 mmHg CO₂ to 85 mm Hg CO₂ or a combination thereof, for less than about 6 minutes, 5 minutes, 4 minutes, 3 minutes, 2 minutes or 1 minute in each hypercapnic stimulation, for a total of 4, 5, 6, 3, or 2 times of stress stimulations to acquire stress images with MRI. In one example, in acquisition of each reference MR image, the PaCO₂ level is the subject's PaCO₂ level at rest in each R; in acquisition of each stress MR image, the PaCO₂ level of the subject is increased by (ΔPaCO₂) at least 25 mmHg, 26 mm Hg, 27 mmHg, 28 mmHg, 29 mmHg, 30 mmHg, 24 mmHg, 23 mmHg, 22 mmHg, 21 mm Hg, or 20 mmHg in each Sᵢ due to administration of the stress agent; the time in each Sᵢ scan is about 6 min, 5 min, 4 min or less, for a total of 4 times, 3 times or 5 times; and the time in each Rᵢ scan can be the same as the time in corresponding Sᵢ scan (e.g., time in R₁=time in S₁; time in R₂=time in S₂; time in R₃=time in S₃ etc; while time in S₁ can be the same or different from the time in S₂).

In other embodiments, the stress agent (e.g., an admixture comprising CO₂) is administered at low doses for longer durations. For example, administering low doses of predetermined amounts of CO₂ for a longer duration comprises administering the predetermined amount of CO₂ at any one or more of about 30 mmHg CO₂ to about 35 mmHg CO₂, about 35 mmHg CO₂ to about 40 mmHg CO₂, about 40 mmHg CO₂ to about 45 mmHg CO₂ or a combination thereof, for any one or more of 10 minutes to 20 minutes, 20 minutes to 30 minutes, 30 minutes to 40 minutes, 40 minutes to 50 minutes, 50 minutes to 1 hour, about 1 to 2 hours, or about 2 to 3 hours, or a combination thereof. In some embodiments, the predetermined levels of CO₂ are administered so that the arterial level of CO₂ reaches the PaCO₂ of any one or more of the above ranges. In one example, in acquisition of each reference MR image, the PaCO₂ level is the subject's PaCO₂ level at rest in each Rᵢ; in acquisition of each stress MR image, the PaCO₂ level of the subject is increased by (ΔPaCO₂) about 5 mmHg, 6 mm Hg, 7 mmHg, 8 mmHg, 9 mmHg, 10 mmHg, 11 mmHg, 12 mmHg, 13 mmHg, 14 mm Hg, or 15 mmHg in each Sᵢ due to administration of the stress agent; the time in each Sᵢ scan is about 10 min, 11 min, 12 min, 13 min, 14 min, 15 min, 20 min, 25 min, 30 min or more, for a total of 4 times, 3 times or 5 times; and the time in each R scan can be the same as the time in corresponding Sᵢ scan (e.g., time in R₁=time in S₁; time in R₂=time in S₂; time in R₃=time in S₃ etc; while time in S₁ can be the same or different from the time in S₂).

In some embodiments, administering a stress agent (e.g., an admixture comprising CO₂) comprises administering the stress agent in a stepwise manner.

In some embodiments, administering the stress agent (e.g., an admixture comprising CO₂) in a stepwise manner comprises administering the stress agent so as to change the PaCO₂ in about 5 mmHg increments in the range of any one or more of 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 70 mmHg to 80 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂.

In some embodiments, administering the stress agent (e.g., an admixture comprising CO₂) in a stepwise manner comprises administering the stress agent so as to change the PaCO₂ in 15 mmHg increments in the range of any one or more of 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 70 mmHg to 80 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂.

In some embodiments, administering the stress agent (e.g., an admixture comprising CO₂) in a stepwise manner comprises administering the stress agent so as to change the PaCO₂ in 25 mmHg increments in the range of any one or more of 20 mmHg to 80 mmHg CO₂, 30 mmHg to 80 mmHg CO₂, 40 mmHg to 80 mmHg CO₂, 50 mmHg to 80 mmHg CO₂, 60 mmHg to 80 mmHg CO₂, 70 mmHg to 80 mmHg CO₂, 20 mmHg to 70 mmHg CO₂, 30 mmHg to 70 mmHg CO₂, 40 mmHg to 70 mmHg CO₂, 50 mmHg to 70 mmHg CO₂, 60 mmHg to 70 mmHg CO₂, 20 mmHg to 60 mmHg CO₂, 30 mmHg to 60 mmHg CO₂, 40 mmHg to 60 mmHg CO₂ or 50 mmHg to 60 mmHg CO₂.

In some embodiments, administering CO₂ comprises administering carbon dioxide in a blockwise manner. In some embodiments, administering an admixture comprising CO₂ comprises administering the admixture comprising CO₂ in a blockwise manner.

In some embodiments, administering CO₂ alters the PaCO₂ in the subject and does not alter the O₂ levels in the subject. In some embodiments, administering the admixture comprising CO₂ alters the PaCO₂ in the subject and does not alter the O₂ levels in the subject. Further embodiments provide that the stress agent is administered while partial pressure of arterial oxygen (PaO₂) of the subject is maintained at a level in a range between 50 mmHg and 150 mmHg. For example, methods include administering CO₂ or an admixture comprising CO₂ so as to change the PaCO₂ of the subject in a range of 20 mmHg to 80 mmHg PaCO₂, 30 mmHg to 80 mmHg PaCO₂, 40 mmHg to 80 mmHg PaCO₂, 50 mmHg to 80 mmHg PaCO₂, 60 mmHg to 80 mmHg PaCO₂, 70 mmHg to 80 mmHg PaCO₂, 20 mmHg to 70 mmHg PaCO₂, 30 mmHg to 70 mmHg PaCO₂, 40 mmHg to 70 mmHg PaCO₂, 50 mmHg to 70 mmHg PaCO₂, 60 mm Hg to 70 mmHg PaCO₂, 20 mmHg to 60 mmHg PaCO₂, 30 mmHg to 60 mmHg PaCO₂, 40 mm Hg to 60 mmHg PaCO₂, or 50 mmHg to 60 mmHg PaCO₂, while partial pressure of arterial oxygen (PaO₂) of the subject is maintained between 50 mmHg and 150 mmHg.

Yet other embodiments provide that PaO₂ level is not intervened during the administration of CO₂ or an admixture comprising CO₂. For example, methods include administering CO₂ or an admixture comprising CO₂ so as to change the PaCO₂ of the subject in a range of 20 mmHg to 80 mmHg PaCO₂, 30 mmHg to 80 mmHg PaCO₂, 40 mmHg to 80 mmHg PaCO₂, 50 mmHg to 80 mmHg PaCO₂, 60 mmHg to 80 mmHg PaCO₂, 70 mmHg to 80 mmHg PaCO₂, 20 mmHg to 70 mmHg PaCO₂, 30 mmHg to 70 mmHg PaCO₂, 40 mmHg to 70 mmHg PaCO₂, 50 mmHg to 70 mmHg PaCO₂, 60 mm Hg to 70 mmHg PaCO₂, 20 mmHg to 60 mmHg PaCO₂, 30 mmHg to 60 mmHg PaCO₂, 40 mm Hg to 60 mmHg PaCO₂, or 50 mmHg to 60 mmHg PaCO₂.

Other increments of carbon dioxide to be administered in a stepwise manner will a readily apparent to a person having ordinary skill in the art.

In some embodiments, administering a stress agent (e.g., an admixture comprising CO₂) comprises administering the stress agent in a blockwise manner. For example, blockwise administration is exemplified in FIGS. 3A and 1A.

In some embodiments the admixture comprising CO₂ or the CO₂ gas is administered via inhalation. The admixture comprising CO₂ or CO₂ may be administered using, for example, RESPIRACT™ platform from Thornhill Research.

Methods of Diagnosis, Prognosis, Selecting Patient Populations and/or Intervention

In some embodiments, the present invention provides a method for performing a cardiac stress test for a subject using an MRI system, which includes (a) directing the MRI system to perform a first sequence that is sensitive to blood oxygenation, blood volume, and/or blood flow, at a cardiovascular system of the subject at a reference partial pressure of arterial carbon dioxide (PaCO₂) level to acquire a first set of reference MR data from the subject; (b) administering an effective amount of a stress agent selected from the group consisting of CO₂ and an admixture comprising CO₂ to attain a stress PaCO₂ level in the subject, wherein the stress agent is administered in an amount effective for inducing hyperemia in the subject; (c) directing the MRI system to perform a second sequence that is sensitive to the blood oxygenation, the blood volume, and/or the blood flow at the cardiovascular system of the subject at the stress PaCO₂ to acquire a first set of stress MR data from the subject; (d) repeating steps (a)-(c) at least once, each repetition generating a subsequent set of reference MR data and a subsequent set of stress MR data; and (e) reconstructing a series of reference images from each set of the reference MR data and registering the series of reference images to obtain a motion-corrected reference image for each set of the reference MR data, thereby obtaining a plurality of motion-corrected reference images from repetition of step (a), and reconstructing a series of stress images from each set of the stress MR data and registering the series of stress images to obtain a motion-corrected stress image for each set of the stress MR data, thereby obtaining a plurality of motion-corrected stress images from repetition of steps (b) and (c).

In some embodiments, the present invention provides a method for performing a cardiac stress test to assess cardiovascular function of a subject using an MRI system, which includes steps (a)-(e), as described above, and step (f) comparing the plurality of motion corrected reference images and the plurality of motion corrected stress images in terms of image voxels or pixels characteristic of the blood oxygenation, the blood volume, and/or the blood flow in at least one region of the cardiovascular system.

In some aspects, an absence of a statistically significant difference in the image voxels or pixels between the plurality of motion corrected reference images and the plurality of motion corrected stress images is indicative of impaired blood oxygenation, impaired blood volume, and/or impaired blood flow, respectively, in the at least one region of the cardiovascular system of the subject.

In some aspects, a presence of a statistically significant difference in the image voxels or pixels between the plurality of motion corrected reference images and the plurality of motion corrected stress images is indicative of hyperemia in response to the stress agent and normal blood oxygenation, normal blood volume, and/or normal blood flow, respectively, in the at least the region of the cardiovascular system of the subject.

Some embodiments of the invention provide a method of detecting the presence or progression of a cardiovascular disease (e.g., associated with impaired blood oxygenation/volume/perfusion) such as myocardial ischemia in a subject using an MRI system, which includes the steps (a)-(f) as described above.

Some embodiments of the one or more methods disclosed herein further includes segmenting the cardiovascular system shown in each motion-corrected reference image and each motion-corrected stress images. Further embodiments provide that in the one or more methods including the step of segmenting, the step of comparing compares at least one segment of the cardiovascular system in the plurality of the motion-corrected reference images with the corresponding segment in the plurality of the motion-corrected stress images, wherein the lack of a statistically significant difference in the image voxels or pixels is indicative of the impaired blood volume, the impaired blood flow, and/or the impaired blood oxygenation in the at least one segment of the cardiovascular system; whereas the presence of a statistically significant difference in the image voxels or pixels is indicative of normal blood volume, normal blood flow, and/or normal blood oxygenation in the at least one segment of the cardiovascular system.

In some embodiments, the MRI sequences are selected from T1, T2, T2*, and ASL.

In some embodiments, the step of comparing the plurality of motion corrected reference images and motion corrected stress images compares blood flow in at least one region of the heart at the reference and stress arterial blood levels of CO₂.

In some embodiments, the step comparing the plurality of motion corrected reference images and motion corrected stress images assesses differences in blood flow as determined by blood oxygenation in the region of the heart.

In some embodiments, the reference arterial blood level of carbon dioxide (CO₂) is for the subject a rest arterial blood level of CO₂.

In some embodiments, the MRI sequences are BOLD-MRI, wherein the BOLD-MRI comprises a free-breathing 3D T2-based sequence at 3T.

In some embodiments, an anova statistical framework is used to compare the plurality of motion corrected reference images and motion corrected stress images.

In some embodiments, the subject at rest is in a normocapnic state.

In some embodiments, the subject is in a hypercapnic state after administration of the stress agent.

In some embodiments, the subject is not administered a contrast agent, radioactive tracer and/or ionizing radiation.

In some embodiments, the CO₂ or admixture comprising CO₂ is administered via inhalation.

In some embodiments, the cardiovascular disease is selected from infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, coronary stenosis, LAD stenosis, and peripheral artery disease.

In some embodiments, the cardiovascular disease is ischemic heart disease.

In some embodiments, the cardiovascular system comprises a heart. In some embodiments, the heart is selected from a whole heart, a portion of the heart, and a section of the heart. In some embodiments, the cardiovascular system comprises a myocardium. In some embodiments, the cardiovascular system comprises at least one coronary artery.

In some embodiments, the blood volume is myocardial blood volume.

In some embodiments, the blood oxygenation is myocardial blood oxygenation.

In some embodiments, the blood flow is myocardial blood flow.

In some embodiments, the method is needle-free.

In some embodiments, the reference arterial partial pressure of carbon dioxide level and the target arterial partial pressure of carbon dioxide level are determined by measuring the end tidal partial pressure of carbon dioxide.

In some embodiments, measured on a breath by breath basis.

In some aspects, the subject in the disclosed methods has respiratory disorders (e.g., asthma, chronic pulmonary disorder, etc.) and/or is suspected of having IHD. In other aspects, the disclosed methods further include selecting, identifying or examining a subject having one or more of respiratory disorders and/or is suspected of having IHD.

In some aspects, the subject suitable for the disclosed methods has renal insufficiency, hence for whom exogenous contrast agents, ionizing radiation or conventional vasodilator stress agents that add renal burden would be unsuitable. In other aspects, the disclosed methods further include selecting, identifying or examining a subject with renal insufficiency, for the disclosed MR imaging or cardiac stress tests.

In some aspects, the subject in one or more of the disclosed methods is a child; or the disclosed methods include selecting or identifying a child for the MR imaging or cardiac stress tests. In further aspects, the subject in one or more of the disclosed methods is a patient who is not a candidate for exercise.

Further embodiments of the invention provide a method of selecting or identifying with a cardiovascular disease (e.g., stenosis; myocardial ischemia), diagnosing a subject of a cardiovascular disease, or examining a subject suspected of having a cardiovascular disease, which includes performing a cardiac stress test using an MRI system in a process as described.

Yet other embodiments of the invention provide a method of diagnosing a subject with a cardiovascular disease and treating the subject, which includes performing a cardiac stress test using an MRI system in a process described above, and prescribing the subject to obtain, or administering, an intervention therapy, a medication and/or a procedure to the subject. Subject diagnosed with a cardiovascular disease can also administer self-care, such as maintaining or adding physical exercise, quitting smoking, and/or consuming low fat diets.

Exemplary treatment for subjects identified or diagnosed with a myocardial ischemia or a coronary artery disease includes medications (e.g., blood thinners such as clopidogrel or aspirin; statin; beta blockers such as atenolol or metoprolol; calcium channel blockers such as amlodipine), angioplasty or bypass surgery.

In some embodiments, the method further comprises selecting a treatment for cardiovascular disease for the subject and/or providing a treatment for cardiovascular disease to the subject and/or administering a treatment for cardiovascular disease to the subject.

For example, an embodiment of the invention provides (a) directing the MRI system to perform a first sequence that is sensitive to blood oxygenation, blood volume, and/or blood flow, at a cardiovascular system of the subject at a pre-defined baseline/reference partial pressure of arterial carbon dioxide (PaCO₂) level or at a rest state to acquire a first set of reference MR data from the subject; (b) administering an effective amount of a stress agent, e.g., selected from the group consisting of CO₂ and an admixture comprising CO₂, attaining a stress PaCO₂ level in the subject, wherein the stress agent is administered in an amount effective for inducing hyperemia or increasing blood velocity and/or flow rate in the subject; (c) directing the MRI system to perform a second sequence that is sensitive to the blood oxygenation, the blood volume, and/or the blood flow at the cardiovascular system of the subject at the stress PaCO₂ level or while the stress agent is administered according to step (b) to acquire a first set of stress MR data from the subject; (d) repeating steps (a)-(c) at least once, each repetition generating a subsequent set of reference MR data and a subsequent set of stress MR data; (e) reconstructing a series of reference images from each set of the reference MR data and registering the series of reference images to obtain a motion-corrected reference image for each set of the reference MR data, thereby obtaining a plurality of motion-corrected reference images from repetition of step (a), and reconstructing a series of stress images from each set of the stress MR data and registering the series of stress images to obtain a motion-corrected stress image for each set of the stress MR data, thereby obtaining a plurality of motion-corrected stress images from repetition of steps (b) and (c); (f) comparing the plurality of motion corrected reference images and the plurality of motion corrected stress images in terms of image voxels or pixels characteristic of the blood oxygenation, the blood volume, and/or the blood flow in at least one region of the cardiovascular system; and (g) selecting the subject for a cardiovascular treatment, and/or administering a treatment to the subject if there is no statistically significant difference in the image voxels or pixels (in one or more regions/segments) between the plurality of motion corrected reference images and the plurality of motion corrected stress images in at least one region of the cardiovascular system; or not selecting the subject for a cardiovascular treatment if there is statistical significant difference in the image voxels or pixels (in all imaged regions/segments) between the plurality of motion corrected reference images and the plurality of motion corrected stress images. In further embodiments, if the subject is not selected or identified as having impaired blood oxygenation, impaired blood volume, or impaired blood perfusion in the heart by the steps described above, the subject is recommended to return for a subsequent cardiac stress testing or other imaging techniques after a period of time. In further embodiments, the subject selected for treatment following the steps above receives a treatment, and is monitored for the treatment efficacy or progression of the cardiovascular disease by being imaged by the described methods/steps (a)-(g) again.

In some embodiments, the method further comprises selecting a preventative treatment for cardiovascular disease for the subject and/or providing a preventative treatment for cardiovascular disease to the subject and/or administering a preventative treatment for cardiovascular disease to the subject.

Various embodiments of the present invention are described in the ensuing examples. The examples are intended to be illustrative and in no way restrictive.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

The invention will be further explained by the following examples, which are intended to be purely exemplary of the invention, and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1. Repeat Hypercapnic Stimulations Increased Statistical Confidence of BOLD-CMR in Assessing Myocardial Oxygenation for Ischemic Heart Disease in Canines

### Materials and Procedures

### Animal Preparation and Method for Inducing Coronary Stenosis

Dogs (N=15, 20-25 kg) were studied with and without surgically induced coronary stenosis. All animals were studied according to the NIH “Guide for the Care and Use of Laboratory Animals” following approval of Institutional Animal Care and Use Committee. In a subset of the animals (stenosis group; N=7), a left lateral thoracotomy was performed. A Doppler probe was attached distal to the first branch of the left anterior descending coronary artery (LAD) to enable measurement of coronary blood flow velocity (CBFV). An externally actuated hydraulic occluder was affixed proximal to the Doppler flow probe. Subsequently, the chest was closed, and the animals were allowed to recover for at least 7 days prior to imaging studies. Prior to all imaging studies, animals were fasted, sedated, intubated and anesthetized. During the imaging studies, anesthesia was maintained with a continuous infusion of propofol. Dogs were transferred to the PET/MR scanner table and were mechanically ventilated through the RESPIRACT™ (Thornhill Research Inc, ON, Canada)). In stenosis studies, coronary stenosis was induced prior to commencing imaging. The perfusion level during rest and stress were confirmed with ¹³NH₃ PET images. Prior to, or right after, the baseline (PETCO₂˜40 mmHg) and peak hypercapnia (PETCO₂˜60 mmHg) BOLD acquisitions, the Doppler transducer (Triton Technology Inc, CA, USA) was connected to the wires originating from the surgically implanted Doppler probe and root-mean-square Doppler flow velocity values were recorded. In dogs where LAD stenosis was to be induced, peak hyperemic coronary blood flow velocity was measured at PETCO₂˜60 mmHg was reduced to coronary blood flow velocity measured under PETCO₂˜40 mmHg to provide a standardized hemodynamically effective constriction that does not decrease blood flow below baseline flow under resting conditions (normocapnia).

### Modulation of Arterial Pressure of CO₂

Prospective targeting of PaO₂ and PaCO₂ was implemented using a validated gas controlling system (RESPIRACT™). In this study, we targeted hypercapnia at PaCO₂=60 mmHg with PaO₂=130 mmHg; and normocapnia at PaCO₂=35 mmHg with PaO₂=130 mmHg. These targets were synchronized with cardiac MR (CMR) and PET acquisitions. Before each image acquisition, PaCO₂ level were stabilized at the targeted level for 1 minute to ensure that target PaCO₂ values were reached. Physiologic response to the stimulations is summarized in Table 1.

### Imaging Protocol

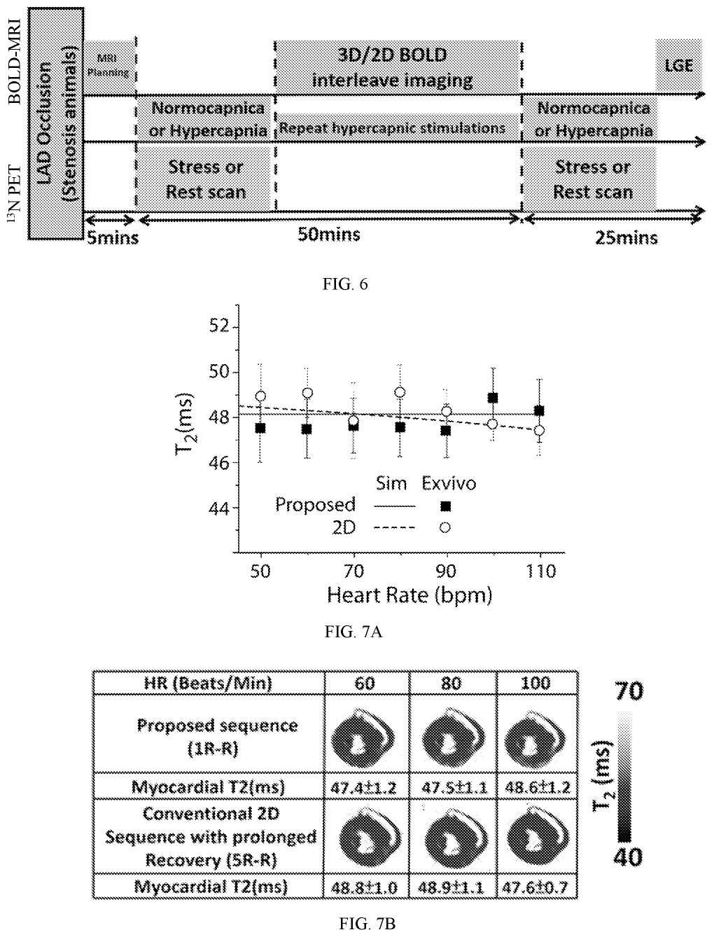

In all imaging studies, ¹³N-ammonia PET and BOLD CMR images were simultaneously acquired using a clinical PET/MR scanner. In animals without coronary stenosis, PET images were acquired under rest and hypercapnia (6 mins) to quantify the MBF under different physiological conditions. A time delay was introduced between sequential PET acquisitions at each physiological condition to ensure sufficient decay of each ¹³N-ammonia dose (5 half-lives, ˜50 minutes). Following the first PET scan, 4 sets of prospectively targeted normocapnia and hypercapnia stimulations were induced using RESPIRACT™. The PaCO₂ levels were maintained for 5 minutes during each physiological state (FIG. 6). BOLD CMR images were acquired 1 minute after reaching the targeted P_{ET}CO₂ level. In animals with coronary stenosis, baseline blood flow prior to surgery was compared to baseline flow post-surgery (on the day of stenosis studies) using ¹³N-ammonia PET. LAD coronary stenoses were induced before the first PET acquisition. Other aspects of the imaging protocol were similar to that implemented in intact animals. A schematic representation of the time course of execution of the study protocol is shown in FIG. 6. During repeat stimulations, two BOLD acquisition methods (2D and 3D T2 maps) were used in a subgroup of animals. In 2D studies (N=5 for both intact and stenosis groups), a conventional 2D T2 mapping sequence was prescribed over a mid-ventricular slice. Images were acquired under short breath holds (<10 s) at 2 minutes and 5 minutes after target P_{ET}CO₂ values were reached. In the 3D acquisitions, the proposed 3D sequence was prescribed under free breathing conditions starting 1 minute after the targeted P_{ET}CO₂ level was reached.

### MRI Pulse Sequence Development

A heart-rate independent, free-breathing, 3D T2 mapping prototype sequence with whole-heart LV coverage, which minimizes the sensitivity to B0 and B1 inhomogeneities, was developed for the PET/MR system. Adiabatic T2 preparation with spoiled gradient-echo (GRE) readout was used to minimize B0 and B1 artifacts that are otherwise prominent at 3T and confound BOLD signal readouts. To improve imaging efficiency and enable data acquisition under free-breathing conditions, a motion-correction platform with a hybrid Cartesian-radial trajectory was applied that permits near perfect imaging efficiency. To further increase acquisition speed and minimize the signal dependence on heart rate between rest and stress, a Saturation Recovery (SR) preparation was integrated with a constant saturation recovery time (T_{SR}) to reset longitudinal magnetization in every heartbeat. To minimize any potential confounding effects associated with differences in T1 recovery following T2 preparation under rest and stress, data was collected and centrically encoded in the through-plane direction. Images were acquired with 3 incremental T2-prepation times (TE=0, 24, 55 ms) and T2 maps were reconstructed using a custom-written Matlab (The Mathworks, Natick, Mass.) script. The accuracy of the proposed approach was studied with computer simulations and ex-vivo tissue preparations. These and the data CMR acquisition parameters are detailed in the Examples section herein.

Assessment of MBF with Simultaneously Acquired ¹³N PET

All PET images were acquired in 3D list mode using ¹³N-ammonia (100 MBq, IV bolus (30 s) followed by 10 cc saline flush) as the blood flow tracer. Prior to each PET scan, MR images were acquired to correct for photon attenuation. A 2-point Dixon MR imaging pulse sequence was used for segmentation and attenuation correction. PET data was acquired over 10 minutes and was started a few seconds before the ¹³N-ammonia injection. In animals without coronary stenosis, images were acquired during hypercapnia and at normocapnia to determine the MBF response in the absence of coronary stenosis. In animals with coronary stenosis, images were acquired at rest and during hypercapnia after infliction of LAD stenosis. The MRI attenuation map and PET images were aligned and adjusted by an experienced technologist. Dynamic PET images were reconstructed with different time periods (twelve 10-s, two 30-s, one 1-min, and one 6-min frames, for a total of 10 min). Images were reconstructed with 3 iterations and 3D post filtering with a 5-mm Gaussian kernel. Data were reconstructed with 2-mm pixels for each dynamic frame. Myocardial blood flow (MBF; ml/min/g) were derived from the PET data using the automated QPET software (Cedars-Sinai Medical Center, Los Angeles, Calif., USA).

### Animal Preparation and Method for Inducing Coronary Stenosis

Canines (N=15, 20-25 kg) were studied with and without surgically induced coronary stenosis. All animals were studied according to the NIH “Guide for the Care and Use of Laboratory Animals” following approval of Institutional Animal Care and Use Committee. In a subset of the animals (stenosis group; N=7), a left lateral thoracotomy was performed. In short, a 20 MHz Doppler probe was attached immediately distal to the first branch of the left anterior descending coronary artery (LAD) to enable measurement of coronary blood flow velocity (CBFV). An externally actuated hydraulic occluder was affixed proximal to the Doppler flow probe. Subsequently, the chest was closed, and the animals were allowed to recover for at least 7 days prior to imaging studies. Animals were monitored post-operatively until they are aware of their surroundings and sternal recumbent. Following surgery, animals received routine post-operative analgesia and were monitored daily for discomfort or distress. Signs of discomfort and/or distress were defined as listlessness, failure to produce stools and/or urine, failure to eat, failure to show usual signs of mobility, and unusual physical symptoms, including redness or swelling of the surgical site. Post-operatively, before each dog awakened, Buprenex (0.1 mg/kg IM) was administered to the animal to alleviate pain and stress. This dosage was continued every 6 hours for 24-36 hours, as indicated by the comfort level of the animal. Antibiotics, Cefazolin (25 mg/kg, IV) was post-operatively administered to animals every 8 hrs for at least 24 hrs. Induction of anesthesia was with Brevital (Methohexital sodium, 11 mg/kg IV), along with pre-anesthetic tranquilizer Innovar (Fentanyl citrate 0.4 mg/ml and Droperidol 20 mg/ml). Prior to all imaging studies, animals were fasted, sedated, intubated and anesthetized with propofol (2.0-5.0 mg/kg, IV). During the imaging studies, anesthesia was maintained with a continuous infusion of propofol (0.03-0.1 mg/kg/min, IV). Dogs were transferred to the PET/MR scanner table and were mechanically ventilated through the RESPIRACT™ (Thornhill Research Inc, ON, Canada). In stenosis studies, coronary stenosis was induced before the imaging protocol. The perfusion level during rest and stress were confirmed with ¹³NH₃ PET images. SPO₂, HR and blood pressure (systolic (SBP), diastolic (DBP), and mean (MAP)) were monitored during the studies. The fluid status during studies was monitored on the basis of continuous pressure measurements and was corrected with saline drip when needed. Prior to, or right after, the baseline (P_{ET}CO₂˜40 mmHg) and peak hypercapnia (P_{ET}CO₂˜60 mmHg) BOLD acquisitions, the Doppler transducer (Triton Technology Inc, CA, USA) was connected to the wires originating from the surgically implanted Doppler probe and root-mean-square Doppler flow velocity values were recorded throughout the cardiac cycles. In dogs where LAD stenosis was to be induced, peak hyperemic coronary blood flow velocity was measured at P_{ET}CO₂˜60 mmHg was reduced to coronary blood flow velocity measured under P_{ET}CO₂˜40 mmHg to provide a standardized hemodynamically effective constriction that does not decrease blood flow below baseline flow under resting conditions (normocapnia). Hence, creation of LAD stenosis was confirmed on the basis of Doppler blood flow and the corresponding reduction in the myocardium was confirmed on the basis of ¹³N-ammonia PET (details below).

### Modulation of Arterial Pressure of CO₂

Prospective targeting of PaO₂ and PaCO₂ was implemented using a validated gas controlling system (RESPIRACT™). The principles of controlling end-tidal gases have been previously described. Briefly, the system is designed to control the arterial blood gas levels using a computerized gas blender and a sequential rebreathing circuit. The algorithm is enabled by equilibrating the rebreathed and alveolar gas with the gas blender from the RESPIRACT™ being used to precisely control the alveolar gas exchange through manipulating the source gases. For example, CO₂ gas exchange follows the relationship that end-tidal CO₂ (P_{ET}CO₂)=V_{CO2}/V_{A}, where V_{CO2} is the volume of CO₂ production by metabolic activity and V_{A} is the alveolar ventilation volume. Since the V_{CO2} can be measured in the exhaled gas, the P_{ET}CO₂ can be targeted by manipulating V_{A}, which is controlled by the gas blender component of RESPIRACT™. Similar considerations are applicable for P_{ET}O₂. These relationships provide the capability to precisely, rapidly and independently controlling P_{ET}O₂ and P_{ET}CO₂, which has been proven to be closely correlated to the PaO₂ and PaCO₂. In this study, we targeted hypercapnia at PaCO₂=60 mmHg with PaO₂=130 mmHg; and normocapnia at PaCO₂=35 mmHg with PaO₂=130 mmHg. These targets were synchronized with cardiac MR (CMR) and PET acquisitions. Before each image acquisition, PaCO₂ level were stabilized at the targeted level for 1 minute to ensure that target PaCO₂ values were reached. Physiologic response to the stimulations is summarized in Table 1.

### Imaging Protocol

In all imaging studies, ¹³N-ammonia PET and BOLD CMR images were simultaneously acquired a clinical PET/MR scanner. In intact group, PET images were acquired under rest and hypercapnia (6 mins) to quantify the myocardial blood flow under different physiological conditions. A time delay was introduced between sequential PET acquisitions at each physiological condition to ensure sufficient decay of each ¹³N-ammonia dose (5 half-lives, ˜50 minutes). The order of PET scans under hypercapnia and normocapnia were randomized. Following the first PET scan, 4 sets of prospectively targeted normocapnia and hypercapnia stimulations were induced using RESPIRACT™ as described in the previous section. The PaCO₂ levels were maintained for 5 minutes during each physiological state (FIG. 6). BOLD CMR images were acquired 1 minute after reaching the targeted P_{ET}CO₂ level. In the stenosis group, baseline blood flow prior to surgery was compared to baseline flow post-surgery (on the day of stenosis studies) using ¹³N-ammonia PET. LAD coronary stenoses were induced before the first PET acquisition. Other aspects of the imaging protocol were similar to that implemented in intact animals. The total span of each imaging study was less than 90 minutes. A schematic representation of the time course of execution of the study protocol is shown in FIG. 6. All stenosis scans were terminated with late gadolinium enhancement (LGE) imaging to rule out infarctions from surgically controlled coronary stenosis. Once the imaging studies were completed, animals were euthanized under anesthesia.

During repeat stimulations, two BOLD acquisition methods (2D and 3D T2 maps) were used in a subgroup of animals. In 2D studies (N=5 for both intact and stenosis groups), a conventional 2D T2 mapping sequence was prescribed over a mid-ventricular slice. Extended trigger pulses (5 R-R) were applied to minimize the heart rate dependency from T1 contamination of T₂ maps. Images were acquired under short breath holds (<10 s) at 2 minutes and 5 minutes after target P_{ET}CO₂ values were reached. Sufficient time between the breath holds were allowed for animals to adapt to the changing P_{ET}CO₂ during the breath holds and ensure stable P_{ET}CO₂ levels. (Normally a R-R interval window determines the percentage variation of the heart rate. Variations of the acquired data outside the window are rejected and not used in the image reconstruction.) In the 3D acquisitions, the proposed 3D sequence was prescribed under free breathing conditions starting 1 minute after the targeted P_{ET}CO₂ level was reached. 3D whole-heart BOLD images were consistently acquired within 4 minutes.

### MRI Pulse Sequence Development

A heart-rate independent, free-breathing, 3D T2 mapping prototype sequence with whole-heart LV coverage, which minimizes the sensitivity to B0 and B1 inhomogeneities was developed for the PET/MR system. Adiabatic T2 preparation with spoiled gradient-echo (GRE) readout was used to minimize B0 and B1 artifacts that are otherwise prominent at 3T and confound BOLD signal readouts. To improve imaging efficiency and enable data acquisition under free-breathing conditions, a motion-correction platform with a hybrid Cartesian-radial trajectory was applied that permits near perfect imaging efficiency. To further increase acquisition speed and minimize the signal dependence on heart rate between rest and stress, a Saturation Recovery (SR) preparation was integrated with a constant saturation recovery time (TSR) to reset longitudinal magnetization in every heartbeat. To minimize any potential confounding effects associated with differences in T1 recovery following T2 preparation under rest and stress, data was collected and centrically encoded in the through-plane direction. Images were acquired with 3 incremental T2-prepation times (TE=0, 24, 55 ms) and T2 maps were reconstructed using a custom-written Matlab (The Mathworks, Natick, Mass.) script.

### Computer Simulations and Ex-Vivo Study

The proposed sequence was tested and validated with a commercially available 2D T2 mapping sequence. To minimize the heartrate dependency of the conventional sequence, an extended recovery time was applied (5R-R) at the cost of increasing scan time by 50%. Computer simulations employing Bloch equations with parameters corresponding to 2D T2 mapping sequence with extended recovery time (5R-R) and proposed 3D T2 mapping approach were performed to assess the accuracy and reproducibility of T2. To validate the simulation findings and to experimentally assess the influence of heart rate on T2 maps, freshly excised canine hearts (n=3) were immersed in saline solution and scanned individually in a whole body clinical PET/MR scanner using a head coil (Siemens Healthcare, Erlangen, Germany) with the aforementioned 2D T2 mapping sequence and proposed 3D sequence. Images were acquired under artificially imposed heart rates of 50-110 beats/min (bpm) in increments of 10 bpm. 3D sequences were prescribed with full-LV coverage and 2D sequences were prescribed to the matched 3D partitions. Heart rates were chosen to capture the typically observed changes between rest and adenosine stress conditions. To minimize bias, prescription the order of the sequences was randomized.

### Data Acquisition Parameters

All MRI studies were performed in a whole-body PET/MR system (Biograph mMR, Siemens Healthcare, Erlangen, Germany). The imaging parameters for all imaging studies and simulations are outlined below:

BOLD CMR Acquisition Parameters—Simulation parameters included myocardial T2=48.5 ms and myocardial T1=1157 ms at 3T. Two T2 mapping sequences (conventional 2D Cartesian bSSFP (2D) and proposed 3D stack of stars GRE (proposed)) were simulated under different heart rates. Transverse magnetization at the center of the k-space was used as the image contrast for each image and was fitted for quantitative T2. Sequence parameters for the CMR acquisitions were:

Conventional 2D T₂ maps—balanced SSFP readouts with Cartesian trajectory. TR/TE=2.9 ms/1.1 ms, iPAT=2, partial Fourier=3/4, flip angle (FA)=35°, BW=1184 Hz/pixel, 86 lines per heartbeat (simulated rate 60/s), trigger pulse=5, FOV=288×360 mm², matrix size=154×192, and voxel size=2.5×1.7×6.0 mm³, T₂ preparation (as TEs)=0, 24, 55 ms.

Proposed 3D T₂ map—GRE readout with stack-of-stars trajectory, TR/TE=3.0 ms/1.5 ms, FA=15°, BW=1100 Hz/pixel, 32 lines per heartbeat, trigger pulse=1, total projections=144/slice, field-of-view (FOV)=380×380×84 mm³, with 14% slice oversampling, matrix size=192×192×16, and voxel size=2.0×2.0×6.0 mm³ interpolated to 1.0×1.0×3.0 mm³, T₂ preparation (as TEs)=0, 24, 55 ms, SR recovery time=130 ms. Gaussian apodization was used to increase SNR.

LGE CMR: Phase-sensitive inversion recovery (PSIR) late-gadolinium-enhancement (LGE) acquisitions were prescribed to rule out infarctions. PSIR LGE images were acquired 10 minutes after Gd-DTPA infusion (0.2 mmol/kg, Gadovist, Bayer Healthcare, Berlin, Germany), using non-selective inversion recovery preparation with GRE readout (TR/TE=3.2/1.5 ms, FA=20°, BW=586 Hz/pixel, matrix=96×192, in-plane resolution=1.3×1.3 mm²; and slice thickness=6.0 mm). A TI-scout sequence was used to find the optimal TI for nulling the healthy myocardium (240-270 ms).

Noninvasive Assessment of MBF with Simultaneously Acquired ¹³N PET

All PET images were acquired using a whole body clinical Biograph mMR (Siemens Healthcare, Erlangen, Germany) in 3D list mode using ¹³N-ammonia (100 MBq, IV bolus (30 s) followed by 10 cc saline flush) as the blood flow tracer. Prior to each PET scan, MR images were acquired to correct for photon attenuation. A 2-point Dixon MR imaging pulse sequence was used for segmentation and attenuation correction. PET data was acquired over 10 minutes and was started a few seconds before the ¹³N-ammonia injection. In intact group, images were acquired during hypercapnia and at normocapnia to determine the MBF response in the absence of coronary stenosis. Specifically, under hypercapnia, PET acquisitions were prescribed 1 minutes after P_{ET}CO₂ reached the targeted level. In stenosis group, images were acquired at rest and during hypercapnia after infliction of LAD coronary stenosis.

### PET Reconstruction and Quantifications of Myocardial Blood Flow (MBF)

The MRI attenuation map and PET images were aligned and adjusted by an experienced technologist. Dynamic PET images were reconstructed with different time periods (twelve 10-s, two 30-s, one 1-min, and one 6-min frames, for a total of 10 min). Images were reconstructed with 3 iterations and 3D post filtering with a 5-mm Gaussian kernel. Two-dimensional attenuation-weighted ordered-subsets expectation maximization was used with standard parameters (3 iterations and 14 subsets and 3D post filtering with a 5-mm Gaussian kernel). Data were reconstructed with 2-mm pixels for each dynamic frame. Myocardial blood flow (MBF; ml/min/g) were derived from the PET data using the automated QPET software (Cedars-Sinai Medical Center, Los Angeles, Calif., USA). Automatic contours were derived for the whole volume of LV using summed dynamic images. The input function was automatically selected in the LV chamber along the long axis. A standard 2-compartment kinetic model for ¹³N-ammonia was used with the dynamic polar maps and flow values (under rest and stress) were derived. All contours were visually checked by an experienced technologist before generating the results.

### PET: Quantification of Myocardial Perfusion Reserve and Identifying Perfusion Deficit

Myocardial Perfusion Reserve (MPR) values were computed as a ratio, by dividing each stress polar map sample by the rest samples at each point. The total MPR (based on hypercapnic (stress) and normocapnic (rest) flows) was computed within the whole LV region bounded by the LV plane. The regional flow was then obtained by dividing the polar map into three regions (left anterior descending artery, left circumflex coronary artery, and right coronary artery) from the standard 16-segment American Heart Association model. Segmental MPR values were extracted automatically for further analysis. The segments with perfusion deficit were identified following the ASNC guideline based on the consensus of 2 observers. Images were scored using 5-point scoring (0=normal perfusion, 1=mild count reduction, 2=moderate count reduction, 3=severe count reduction, 4=absent uptake). Diseased segments were identified using visual scores 3 as the cutoff.

### BOLD CMR Analysis

All BOLD CMR images were analyzed with custom scripts written in Matlab. For 2D image data sets, BOLD CMR images (T2 maps) were first registered to the initial normocapnic image using a non-rigid registration tool from an open source image registration toolbox (ANTs). Endocardial and epicardial contours were traced to delineate the myocardium from the mid ventricle 2D images and segmented according to the AHA recommendation. Mean T2 of each segment was calculated for images acquired during the repeat stimulations. At each physiological state, % BOLD Response was computed as the T2 values acquired under each time point normalized by the mean T2 value acquired under normocapnia, multiplied by 100%. Time resolved mean % BOLD Response were derived for each of the six segments. Signal averaging was performed over segmental T₂ values.

For 3D image data sets, similar registration and contouring processes as in 2D studies were performed on the whole LV volume. The myocardium was segmented according to AHA 16 segmentations. BOLD signal from each segment was extracted for each physiological state and separated into hypercapnia and normocapnia states according to the P_{ET}CO₂ level at acquisition. Subsequently, segmental BOLD signals from each segment was concatenated across the number of stimulations. Myocardial BOLD signals acquired under the 2 states were compared using the statistical model as described in Results. Regional p-values were projected onto an averaged 3D segmentation model for visual representation. Sensitivity, specificity and accuracy of the proposed method were evaluated using p<0.05 as the cutoff for disease and ¹³N-Ammonia PET as the gold standard. Sensitivity was defined as number of segments with true positive disease assessments divided by number of all segments with positive disease assessments. (Sensitivity=TP/(TP+FN)). Specificity was defined as number of segments with true negative disease assessments divided by number of all segments with negative disease assessments. (Specificity=TN/(TN+FP)) and Accuracy was defined as number of segments with correct assessments divided by number of all segments. (Accuracy=(TN+TP)/(TN+TP+FN+FP)).

### Statistical Modeling and Analysis of Myocardial BOLD Response

Rest BOLD signals were generated for reach segment with Gaussian distribution with the mean (μ) and standard deviation (δ) reported in previous studies acquired using cardiac 2D T2 mapping. Stress signals were generated randomly based on the rest signal and BOLD response (μStress signal=μRest signal+μBOLD response (1% to 20%)) and the number of repeat-measurements (1 to 30). BOLD signals from each segment were grouped into rest and stress states and compared using repeat measurement ANOVA test which tested the hypotheses:

H₀ [Null: BOLD response absent]: T2 during normocapnia=T2 during hypercapnia H₁ [Alternate: BOLD response present]: T2 during normocapnia≠T2 during hypercapnia.

Each test was repeated 200 times and averaged p-values were reported for each peak BOLD response and number of measurements. Sequential Holm-Bonferroni corrections were used to adjust for multiple measurements.

The BOLD images acquired under different physiological states were first segmented according to the AHA 16 segmentation model and were used as multiple samples to test the null hypothesize described in previous section. Repeat measurement ANOVA tests were used to compare cumulative BOLD signals from each segmental position with for increasing stimulation pairs 1 to 4. Sequential Holm-Bonferroni corrections were used to adjust for multiple measurements. Significance was set at α=0.05.

### Experimental Results

### Repeat Stimulation for Enabling Robust Myocardial BOLD MRI: Proof-of-Concept Using 2D BOLD MRI.

Previous studies have shown that arterial CO₂ tension when increased by 25 mmHg from baseline levels can accentuate myocardial blood flow by more than 2-fold, a hallmark of potent coronary vasodilators; and that such changes can be identified with 2D myocardial BOLD-CMR. However, studies to determine whether repeat exposure of the heart to a predefined CO₂ stimulus can be used to improve the detection of healthy and hypoperfused myocardium with or without coronary stenosis have not been reported. To fill this gap in knowledge, we studied this in two parts. First, we performed experiments in dogs exposed to repeat hypercapnic stimulus (normocapnia, end-tidal CO₂ (P_{ET}CO₂)=35 mmHg; and hypercapnia P_{ET}CO₂=60 mmHg) and free-breathing 2D BOLD-CMR to assess whether the repeat stimulations can identify healthy myocardium in canines without coronary stenosis when the resulting BOLD responses are averaged. Next, we performed studies in dogs to identify whether ischemic territories can be identified following repeat hypercapnic stimulation (normocapnia P_{ET}CO₂+35 mmHg; and hypercapnia P_{ET}CO₂=60 mmHg) and 2D BOLD-CMR on the basis of signal averaging to reduce image noise. We validated our findings against simultaneously acquired ¹³N-ammonia PET.

Typical results from healthy dogs (i.e. without coronary artery stenosis) exposed to intermittent hypercapnia (established with prospective control of PaCO₂) in an animal are shown in FIG. 1B. To investigate the dynamic myocardial BOLD response as a function of PaCO₂ in each myocardial segment, we acquired BOLD images of the mid-ventricular myocardium and segmented images according to the American Heart Association (AHA) 6-segment model and measured the BOLD response in each segment. Every segment showed elevated BOLD response during the hypercapnic stimulations that were absent during normocapnia (FIG. 1B). When this was repeated (i.e. animals were subjected to repeat hypercapnia and normocapnia), the pattern of BOLD response was reproducible across all segments. Average myocardial BOLD response derived following multiple stimulations was more homogeneous and higher in magnitude compared to a single stimulation. This observation was consistent with myocardial blood flow changes observed with ¹³N-ammonia PET.

To examine whether this approach could be used to improve the identification of myocardial territories subtended by a significant coronary stenosis (˜50% narrowing), we surgically controlled the left-anterior descending coronary artery (LAD) diameter by adjusting the doppler flow velocity of the vessel. Subsequently we exposed each animal to a single and multiple PaCO₂ stimulations during which time, 2D BOLD-MRI and ¹³N-ammonia PET scans were acquired. BOLD response observed in each segment of the mid-ventricular myocardium (segmented according to the AHA recommendation) was measured. Typical BOLD response that we observed in an animal is shown in FIG. 1C. Myocardial BOLD response to hypercapnia was strong in segments 1 to 5, but not in segment 6 (FIG. 1C). Maps of BOLD response observed following single PaCO₂ stimulation was relatively heterogeneous (FIG. 1D, left), but the average BOLD response following four repeat stimulations showed a confined region of impaired BOLD response consistent with the LAD territory (FIG. 1D, middle), which was consistent with ¹³N-ammonia PET (FIG. 1D, right).

Fast, Free-Breathing, Whole-Heart Myocardial BOLD MRI with Repeat Hypercapnic Stimulations for Accurate and Objective Identification of Myocardium Perfused by Healthy Coronary Arteries.

While repeat hypercapnic stimulations combined with 2D BOLD-CMR and signal averaging can significantly improve the visualization of standard, single-stimulation, BOLD-CMR, it has practical limitations. First, with this approach the BOLD responses need to be visually delineated which introduces subjectivity into image analysis. Second, 2D CMR acquisition schemes are limited by (a) inadequate speed to fully image the heart multiple times to accommodate repeat hypercapnic stimulations; (b) irrecoverable cardiac motion between multiple acquisitions leading to misregistration errors; and (c) undesirable contributions from T1 weighing, coil bias, breathing motion and heart rate dependency, all of which confound the BOLD response. Collectively, these limitations can compromise both sensitivity and specificity of BOLD-CMR. We addressed these key limitations in two steps.

In the first step, we performed numerical simulations by considering a range of peak BOLD signal responses and associated noise, to estimate whether the confidence in detecting myocardial BOLD response can be improved by increasing the number of measurements in a statistical test employing repeated measures Analysis of variance (ANOVA). The results (FIG. 2) showed that the statistical confidence (p<0.05) in identifying the presence of a BOLD response is directly related to the maximal dynamic range of the response available and the number of repeat measurements. Specifically, it identified that for a given average myocardial BOLD response in the heart, typically ˜10% (24), more than 3 repeat measurements would be needed to objectively identify the healthy myocardial territories. This model provides the basis for developing a statistical framework for objectively discriminating between myocardial regions that are responsive to a given stimulus from those that are not on the basis of repeat measurements.

To address the second limitation, we developed a fast free-breathing 3D T₂ mapping technique at a magnetic field strength of 3T that is insensitive to heart-rate changes between rest and stress states, which would support repeat imaging of the whole heart under multiple hypercapnic/normocapnic stimulations. We then performed in-vivo studies in healthy canines under repeat hypercapnic/normocapnic stimulations. Subsequently we analyzed the observed BOLD response within an ANOVA framework to derive statistical parametric maps of p-values.

The framework of the data acquisition protocol, image acquisition and reconstruction strategy, and statistical analysis used to analyze the BOLD images is summarized in FIGS. 3A and 3B. The data acquisition protocol under time-varying PaCO₂ (i.e. alternating between normocapnia and hypercapnia) is shown in FIG. 3A. The confounder-corrected T₂ CMR pulse sequence (magnetization preparation, time-efficient k-space sampling, motion-corrected T₂ mapping) that we developed for rapidly imaging the whole-heart under hypercapnic stimulation is shown in FIG. 3B. We used the T₂ values to assess the BOLD response. FIG. 3C shows representative whole-heart BOLD response from a dog under a single hypercapnic/normocapnic stimulation using the new imaging sequence. A mid-ventricular BOLD response from the same animal acquired under similar conditions, using the standard short breath-held 2D BOLD-CMR, is shown for reference. The myocardial BOLD response from the 2D and 3D approaches were similar, albeit both approaches showed significant heterogeneity of response. FIG. 3D shows the statistical framework we used to identify the healthy myocardial territories. To achieve this, animals were subjected to repeat hypercapnic/normocapnic stimulations. The whole-heart T₂ images at each state of PaCO₂ acquired under hypercapnic/normocapnic conditions were registered to the initial 3D myocardial T₂ maps acquired under normocapnia using non-rigid registration (Advanced Normalization Tools, ANTs). Subsequently, animals underwent repeat hypercapnic/normocapnic stimulations. The whole-heart images were segmented according to the recommendation of AHA. Segmental myocardial T₂ values acquired under normocapnia and hypercapnia were compared using all the segments after each stimulation block (hypercapnia and normocapnia pair) with ANOVA statistics to test the null hypothesis:

H₀ [Null: BOLD response absent]: T₂ during normocapnia=T₂ during hypercapnia H₁ [Alternate: BOLD response present]: T₂ during normocapnia≠T₂ during hypercapnia.

Null hypotheses were rejected when p<0.05. The segmental p-values from repeat measurement one-way ANOVA were used to create statistical parametric maps (SPM) as shown in FIG. 3D. In these maps, myocardial segments with p<0.05 were the segments showing statistically significant BOLD response to hypercapnia following one or more hypercapnic stimulation(s).

Using this approach, we studied healthy dogs (N=8) exposed to intermittent hypercapnia. A representative case from this study is shown in FIG. 4A, where the segmental p-values were mapped following each stimulation as SPM. Note that, although there is marked heterogeneity in BOLD response following a single stimulation, with each repeat stimulation, the statistical confidence in observing a BOLD response increased and became homogeneous across the heart. A direct comparison between the results in FIG. 4A, averaged across all AHA segments as a function of number of stimulations, is shown along with ¹³N-ammonia PET map of myocardial perfusion reserve (MPR) in FIG. 4B. The mean and standard-deviation of the p-values observed following each stimulation derived across all myocardial segments of the heart decreased with each repeat stimulation as shown in FIG. 4B, left. On the basis of PET images, the MPR derived under hypercapnia and normocapnia in the same animal and imaging session confirmed the absence of perfusion deficits and uniform vasodilatory response across the left ventricle (FIG. 4B, right). The collective findings of SPM across all animals, following single and quadruple stimulation blocks, along with mean ¹³N-Ammonia PET MPR are shown in FIG. 4C, respectively. Following a single stimulation, there is marked heterogeneity in p-values suggesting that healthy myocardium can be mischaracterized to be non-responsive likely due to dominance of noise over small BOLD signal change; however, with repeat stimulation, the noise and thus the errors, are significantly reduced. These results support the notion that when repeat hypercapnic stimulations are combined with confounder-corrected fast 3D BOLD-MRI, it is possible to substantially increase the confidence in detecting healthy myocardial territories without contrast agent or ionizing radiation at levels that are realized with the gold-standard, ¹³N-ammonia PET.

Accurate Detection of Myocardial Segments Subtended by Clinically Significant Coronary Stenosis with Statistical Parametric Maps Derived Using 3D Myocardial BOLD MRI and Repeat Hypercapnia.

In the previous section, we demonstrated that BOLD response can be accurately detected in healthy myocardium using an approach which integrates the results from a fast 3D technique, repeat hypercapnic stimulation, to arrive at statistical parametric maps. However, whether this approach can be used to identify myocardial territories affected by a functionally significant coronary stenosis is not known. To address this gap and test whether our approach can be extended for identifying reversible perfusion defect territories, we performed additional studies in dogs with non-flow limiting LAD coronary stenosis. These animals underwent repeat stimulations (4 blocks; each block consisting of hypercapnia and normocapnia) and whole-heart BOLD images were acquired at each of the hypercapnic and normocapnic states in a similar manner to FIG. 3A. Hearts were registered using ANTs, described in B. B. Avants, Neurolmage, 54, 2033-2044 (2011), and the myocardium was segmented according to the AHA recommendation. The statistical framework was applied using the same hypothesis tests as outlined in the previous section to identify remote (i.e. unaffected/healthy) myocardial territories. The p-values were then used to construct SPMs of the heart.

The findings from this study are summarized in FIG. 5A-5D. FIG. 5A from a representative animal with LAD stenosis shows the myocardial SPM along the long- and short-axis orientations of the heart, along with standard bull's eye representation based on AHA segmentations. Note the significant heterogeneity in BOLD response throughout the myocardium following single stimulation and the convergence of two myocardial territories with increasing number of stimulations. The spatial localization of these territories was visually concordant with the ¹³N-ammonia PET MPR (FIG. 5B) following repeat stimulation. The segmental territories identified to be remote and affected based on ¹³N-ammonia PET MPR showed distinct statistical characteristics. For the case in FIG. 5A, mean and standard-deviation of the p-values of all remote territories were significantly lower than the affected territories independent of the number of stimulations. Notably the p-values of the remote territories quickly converged to low values after the second hypercapnic stimulation and reached statistical significance by the fourth hypercapnic stimulation. However, the affected territories retained high p values and were heterogeneous despite the increasing number of stimulations. These observations were consistent with the spatially observed differences in MPR based on ¹³N-ammonia PET and consistent across all animals (FIG. 5C). We found that regardless of the number of stimulations, nearly all measurements showed a sensitivity that is >80% in identifying the affected myocardium. However, the specificity for identifying healthy myocardium was only 36% with a single stimulation and with each additional stimulation, the specificity increased significantly, reaching 92% following the fourth stimulation. A similar observation was evident with accuracy; while the accuracy after a single stimulation was 49%, with each increasing stimulation, the accuracy increased substantially and reached 91% following the fourth stimulation. These results support the notion that a statistical parametric mapping, which is enabled by repeatedly stimulating the heart with prospective control of the PaCO₂ and fast, 3D whole-heart T₂ mapping, can markedly increase the accuracy of BOLD-CMR for identifying hypoperfused myocardial territories to levels observed with ¹³N-ammonia PET.

Accurate identification of myocardial territories affected by coronary artery disease is critical for managing patients with ischemic heart disease. Current methods used for this purpose however, require ionizing radiation or exogenous contrast media. In the best case, these methods expose patients to incremental risk; and in the worst case, they are contraindicated. Previous efforts to address these limitations, particularly on the basis of myocardial BOLD-CMR, have made important progress; however, clinical adoption of it remains uncertain due to limited reliability. In this work, we demonstrated how it is possible to overcome this key obstacle through a new approach, which identifies the affected and healthy/remote myocardial territories using a statistical framework. This is enabled by intermittent hypercapnia to repeatedly stimulate myocardial blood flow and rapid, free-breathing whole-heart T₂ mapping to acquire BOLD images, and a computational platform to perform motion-corrected registration and segmentation. Our investigation, performed using a clinically relevant animal model, systematically demonstrates through a set of progressively advancing studies how this can be accomplished. In the first study, using a 2D T₂ CMR with limited spatial coverage (single, short-axis slice), we show that repeat modulation of myocardial blood flow changes in the heart with hypercapnia allows visual identification of (a) the healthy myocardium in animals without coronary stenosis; and (b) the affected and remote myocardial segments in animals with coronary stenosis. To overcome the spatial coverage and registration limitations inherent to the 2D approach, we developed a time-efficient, confounder-corrected, whole-heart T₂ mapping that can be performed under free-breathing conditions. We then applied this imaging approach with rapid prospective control of PaCO₂ to generate whole heart myocardial BOLD images under hypercapnia and normocapnia. These datasets were then registered together and analyzed segmentally in a statistical framework to demonstrate that SPMs can be generated to accurately identify the healthy myocardium in animals without coronary narrowing. Finally, we extended the approach in animals with controlled coronary artery stenosis so that it can be used to objectively identify healthy and affected myocardium in the setting of clinically significant coronary stenosis with unprecedented sensitivity, specificity and accuracy; all above 90%.

This Example assessed segmental changes in myocardial perfusion based on the changes in myocardial oxygenation associated with clinically significant coronary stenosis. While this is sufficient to meet the current clinical need in the setting of coronary artery disease, expanding this approach to pixel-wise assessment of myocardial oxygenation would open the door for testing novel physiological hypotheses surrounding IHD that are yet to be proven. For instance, pixel-wise cfMRI could be used to evaluate alterations in microcirculatory oxygenation, which could empower the assessment of microvascular disease, where the myocardial blood flow to the subendocardium is believed to be impaired even in the absence of occlusive coronary disease. Current methods do not have the capacity to confirm or refute this since the available methods rely on washout kinetics of the contrast medium and not oxygenation. Hence, pixel-wise assessment of myocardial oxygenation enabled by cfMRI can be instrumental for accurately discerning whether the transmural changes in blood flow and oxygenation occur in parallel. Such an understanding could provide new insights that can improve our understanding of how angina develops in patients with microvascular disease and evaluate therapies to alleviate microvascular impairments in oxygenation. Studies of this nature are likely to demand more advanced segmentation and registration approaches so that pixel-wise analysis can be accurately performed. We anticipate that these demands can be met with segmentation and registration algorithms that are actively being developed for cardiac image analysis. Additionally, to enable pixel-wise SPM, additional studies would be required to determine the number of minimum stimulations necessary for accurate assessment of BOLD signal changes at the pixel level.

There are multiple other conditions where cfMRI could be useful as well. cfMRI identifies the affected regions of the myocardium as those regions that do not respond to repeat hypercapnic stimulation. Although we used this approach to identify territories affected by stenosis of a single coronary vessel, we anticipate that this approach can be applied to other patterns of coronary artery disease as well. Specifically, this approach may be extended for identifying clinically significant multi-vessel coronary artery disease, which is believed to result in balanced ischemia. In addition, cfMRI may also be used to examine changes in myocardial oxygenation of non-ischemic origin, such as hypertrophic heart disease, which is known to impair myocardial oxygenation reserve. Moreover, although our studies showed that cfMRI can identify substantially reduced blood flow and oxygenation, identification of early changes in myocardial oxygenation (e.g., from subclinical level of coronary stenosis or early changes in the heart due to hypertrophy) would require additional studies. We anticipate that these studies would benefit from refined statistical hypotheses and/or selection of optimal statistical thresholds that build on identifying myocardial territories of interest based on cfMRI.

Finally, given the lack of invasive or non-invasive methods to directly assess myocardial oxygenation in vivo, we demonstrated the capacity of cfMRI to accurately identify myocardial territories affected by coronary stenosis on the basis of ¹³N-Ammonia PET under identical physiological conditions. Nonetheless, the tapering off in sensitivity and specificity between cfMRI and ¹³N-Ammonia PET at ˜90% can be suggestive of the potential differences between flow and oxygenation. Further studies would be needed to probe the conditions under which myocardial oxygenation and flow changes are congruent or different.

Other methods to assess IHD without contrast agents or ionizing radiation are under development or are emerging. Amongst these, spectroscopic CMR approaches have the capacity to offer insight into myocardial oxygenation, but they have not been successfully translated into clinical practice due to poor reliability. One recent study has studied the capacity of native T1 CMR to identify IHD patients without exogenous contrast agents or ionizing radiation. Randomized multi-center studies to evaluate the capacity of native T1 CMR for diagnosis of IHD in a spectrum of patients presenting with the disease is likely needed.

Tolerable levels of hypercapnia lead to more than 2-fold increase in myocardial blood flow and similar modulation in oxygenation in extent to that observed with adenosine (commonly used coronary vasodilator) in both in dogs and in humans. To date, hypercapnia has been shown to be safe and tolerable in a broad spectrum of patients (age range 9 to 88). Although this study is on canines, given that all cardiac stress testing paradigms have been first successfully demonstrated in dogs, and 25-mmgHg increase in PaCO₂ is tolerable in humans, we believe the described methods and approach would translate well in humans.

Given the growing availability of MRI systems, the infrastructure costs required to translate the proposed approach into the clinical settings, which already have access to MRI suites, is expected to be minimal (costing less than 1% of the total cost of the scanner environment). Further, since the proposed strategy does not require contrast agents, infusion pumps etc., it would yield substantial cost savings to the medical centers relative to the status quo. Although, a direct translation of the proposed approach in the current state is expected to take ˜40 minutes in human subjects, compared to the ˜10-15 minutes of imaging duration with standard methods, methods that can reduce scan time (for e.g., through faster data acquisitions taking advantage of spa ti o-temporal redundancies in conjunction with utilization of generalized linear models for signal analysis), are expected to permit the proposed approach to be executed within the standard duration of cardiac stress tests. This would also limit the hypercapnic durations to a much shorter time (<4 minute).

Although a 25-mmHg increase in PaCO₂ is expected to be tolerable by most people, some may find it uncomfortable, which may be addressed by taking advantage of the flexibility cfMRI offers for fine tuning image acquisition and analysis. For example, in patients who could tolerate only a lower hypercapnic stimulus, a greater number of weaker hypercapnic stimulations may offer a viable alternative. When this is combined with accelerated data acquisition strategies to yield images of higher temporal resolution to deploy advanced statistical methods that automatically find thresholds of affected regions in a multivariate fashion, it may be possible to identify myocardial territories supplied by stenotic coronary arteries, even in subjects with lower tolerance for hypercapnia. Alternatively, in patients who can tolerate a 25-mmHg of stimulus but cannot tolerate multiple repeat stimulations of it, an alternative may be rapid acquisition of images under other waveforms of PaCO₂ (e.g. ramps instead of blocks or shorter frequency with longer duration of hypercapnia), which are all possible with the proposed prospective control of PaCO₂.

The demonstrated approach opens the door to new opportunities for cardiac stress testing in some of the most vulnerable patients. First, cardiac stress testing may be enabled in adult patients with renal insufficiency, who would otherwise receive multiple doses of ionizing radiation, which can expose them to greater risks associated with radiation. It also offers an alternative to patients who are not candidates for exercise or intravenously administered vasodilatory agents as part of stress tests. Further, it allows cardiac stress testing in the children without ionizing radiation, contrast agents, pharmacological stress or needles. The demonstrated methods not only improve existing care but also permits management of IHD in those who are contraindicated for standard cardiac stress tests.

Methods are provided using cfMRI for non-invasive determination of healthy myocardium and myocardium affected by reversible perfusion defects due to coronary stenosis on the basis of myocardial oxygenation with unprecedented reliability. This integrated approach has the capacity to open a new paradigm for a radiation-, contrast- and needle-free approach for accurately determining reversible perfusion defects in patients suspected of having functionally significant coronary artery disease. Further, it has the desirable characteristics to access multiple other myocardial pathologies on the basis of oxygenation.

TABLE 1
Physiological Parameters During Normocapnia and Hypercapnia

| | Normocapnia | Hypercapnia | |
|---|---|---|---|
| PaCO₂ (mmHg) | 37 ± 2 | 61 ± 2* | (p < 0.01) |
| PaO₂ (mmHg) | 131 ± 5 | 133 ± 3 | (p = 0.2) |
| Heart Rate (beats/min) | 70 ± 15 | 83 ± 18* | (p < 0.05) |
| Systolic Blood Pressure | 121 ± 20 | 1223 ± 19 | (p = 0.3) |
| (mmHg) | | | |
| Diastolic Blood Pressure | 67 ± 19 | 69 ± 18 | (p = 0.2) |
| (mmHg) | | | |
| Mean Arterial Pressure | 80 ± 19 | 83 ± 18 | (p = 0.1) |
| (mmHg) | | | |
| Rate Pressure Product | 5674 ± 2042 | 6948 ± 2320 | (p < 0.05) |

### Example 2. Reduced Hypercapnia Duration with Fast, Highly-Time-Resolved, Whole-Heart, Free-Breathing, Confounder-Reduced BOLD-CMR to Assess Ischemic Burden

cfMRI may require long hypercapnia duration (˜24 min) for repeated stimulations. Herein we propose a strategy for completing hypercapnia under 6 min, which is comparable to the standard duration of stress period. We conceive to (a) develop a fast, highly time-resolved BOLD CMR approach that minimizes the hypercapnia exposure to <6 minutes; (b) validate this approach in an animal model with controllable coronary stenosis; and (c) assess its feasibility in IHD patients.

We use a generalized linear model (GLM) analysis: A linear model, y(t)=ax(t)+β+e(t), will be used to relate the stimulus (hypercapnic PaCO₂) and a response (T2 values); where x(t) and y(t) are time-dependent vectors of stimulus and response. x(t) will be delivered as a normocapnic and hypercarpnic paradigm (square wave) via RESPIRACT. PaCO₂ will be modeled as a piece-wise constant function with values of either 0 (normocapnia) or 1 (hypercapnia). a is a parameter estimate of x(t) that conditions the system to obey the linear model. β is a column vector that corresponds to values of y(t) at baseline, i.e., at the particular times when x(t)=0; e(t) is a column vector of the error in the model fitting. The time series of BOLD responses from repeat PaCO₂ stimulation will be fit to the model data on a voxel-by-voxel basis, and an estimate on the significance of α will be used to derive statistical parametric maps (SPM).

### Example 2-1. To Develop a Whole-Heart, Highly Time-Resolved, Free-Breathing, Confounder-Reduced 3D T2 BOLD CMR Under Prospectively Controlled PaCO₂ Modulation that can be Completed in Less than 12 Mins

Compared to other approach that takes nearly 48 minutes to complete (24 minutes of hypercapnia), herein we accelerate imaging by at least 5-fold without compromising the signal-to-noise ratio (SNR). At the same time, we use statistical and image processing methods to post-process the data to estimate hyperemic volumes. To do this we develop a highly time-resolved dynamic whole-heart 3D T2 mapping approach by taking advantage of temporal sparsity and compressive sensing; analyze the temporal data using GLM; and optionally validate our findings against simultaneously acquired ¹³N-PET.

Preliminary data indicates heart-rate changes between rest and stress acquisitions can impair T2-based BOLD CMR. Segmented acquisitions with fixed R-R intervals that result in insufficient T1 recovery between readouts can lead to heart-rate (HR) dependent signal differences, from which most conventional BOLD methods may suffer.

Herein we developed a free-breathing SR-prepared whole-heart 3D T2 method that is HR independent. We tested this in healthy animals (n=10) and compared it to a free-breathing whole-heart 3D T2 mapping approach that does not account for HR variations (AHR) and validated our findings with ¹³N-PET in a PET/MR scanner. Mean myocardial SNR of T2 maps at rest was 22±3. HR-independent T2 CMR showed significantly greater hyperemic response than 3D T2 maps that did not account for ΔHR and showed better alignment with PET (FIGS. 8A and 8B). The loss of BOLD contrast (indexed as a relative difference between HR-independent T2 and HR-dependent T2) was highly correlated with ΔHR between rest and stress (FIG. 8C). Hence, HR-independent sequences can mitigate BOLD sensitivity reductions.

Further preliminary data indicates contrast-to-noise ratio (CNR) estimates for T2 Golden-angle RAdial Sparse Parallel (GRASP) CMR.

Accelerating data acquisition to capture a single image can result in amplification of noise; but dynamic imaging strategies, such as GRASP, that rely on compressive sensing (CS) reconstruction, which exploit temporal redundancy to reconstruct images, can minimize noise amplification. We further looked at the relation between CNR, image sharpness and temporal resolution (TRes, i.e., extent of undersampling) in native T2-w CMR.

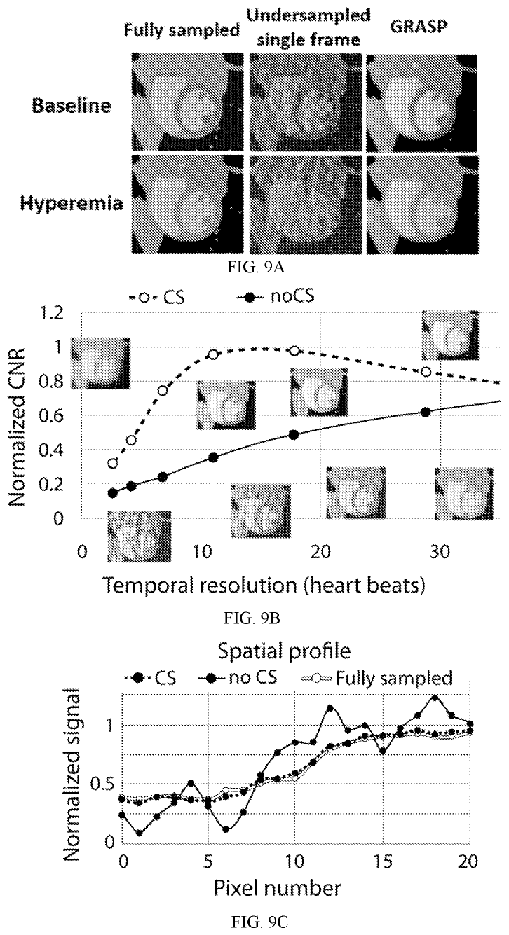

We estimated the relative CNR at various TRes using a numerical cardiac MR phantom (MRXCat, ETH Zurich). 3D stack-of-stars images with various TRes (8-120 heart beats; 7 spoke/beat) were reconstructed with and without CS. Relative myocardial CNRs (normalized to the myocardial signal difference in the fully sampled image between baseline and hyperemia) and a representative signal profile at blood-myocardial interface were computed. Typical images reconstructed with and without CS (TRes of 11 heart beats) are shown in FIG. 9A. Note that at TRes of 11 heart beats, GRASP reconstruction yields similar image quality and CNR compared to the fully sampled images. The relation between relative myocardial CNR and TRes is shown in FIG. 9B. Note that (a) for TRes between 18 to 20 heart beats, images can be reconstructed with minimal CNR loss using GRASP, which offers a suitable tradeoff between temporal smoothing and CNR; and (b) GRASP approach with TRes of 11 beats is nearly 4-fold faster than the conventional (single frame) approach. Normalized signal profile of the myocardium/blood interface is demonstrated in FIG. 9C, which shows that the slope and sharpness of the signal profile at the interface in CS reconstructed image is comparable to the fully sampled image. The temporal regularization, however may lead to motion blurring in in-vivo acquisitions, which would be addressed by the motion-correction framework. Hence, T2-w GRASP, using CS, can achieve a 4-fold image acceleration with preserved CNR and sharpness.

Preliminary Data on Highly Temporally Resolved Non-Contrast 3D Whole Heart GRASP CMR:

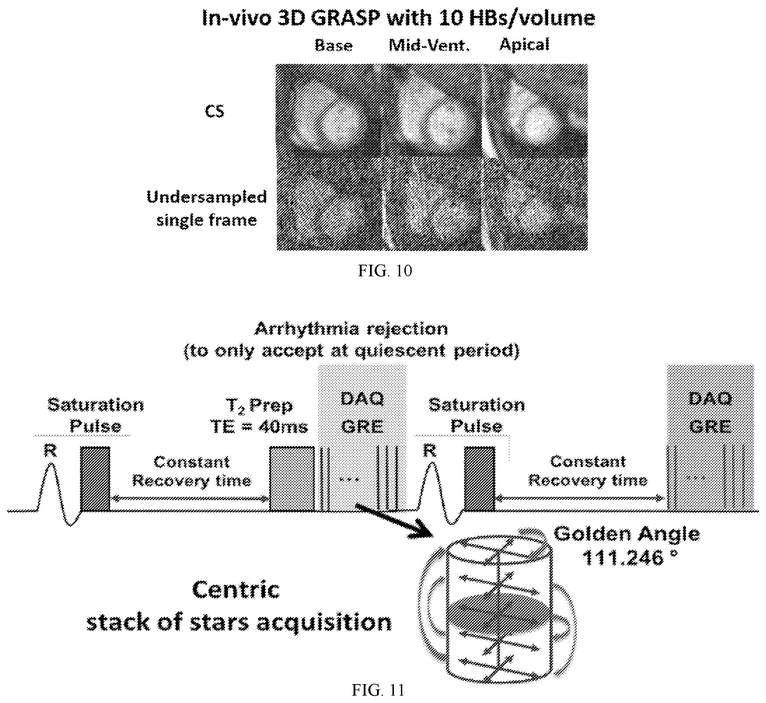

ECG-gated images were continuously acquired for 6 mins. using stack-of-star GRE readouts with golden ratio radial trajectory. Data were reconstructed using temporal resolution=10 heartbeats/volume with and without CS regularization. Typical short-axis images (FIG. 10) show that image quality of is significantly improved with CS, permitting good image contrast between blood and myocardium with minimal spatial blurring. Hence, even in the absence of contrast enhancement, GRASP reconstruction can significantly improve image quality and SNR in images acquired with a temporal footprint of 10 heartbeats.

Pulse sequence Development: Dynamic, time-efficient, HR-independent, T1- and arrhythmia-insensitive, whole-heart, free-breathing T2 maps under prospective control of PaCO₂ with hypercapnic blocks lasting <1.5 minutes.

(a) Optimized Data Acquisition (FIG. 11): K-space data will be acquired over mid diastole with interleaved adiabatic T2 preparation with GRE readout that is synchronized with RESPIRACT. A centric encoded stack-of-stars trajectory with a golden-ratio increment (spatial resolution: 1.5×1.5×6 mm³; in-plane matrix: 144×144) with arrhythmia rejection will be used. To eliminate heart-rate dependency and accelerate data acquisition, an SR preparation pulse will be applied to reset M_{z} magnetization in each heartbeat. Two T2 contrasts (TE=0 or 40 ms), previously optimized for BOLD CMR, will be acquired in an interleaved fashion. Assuming an LV cavity ˜70 mm long (12 slices), at a mean HR of 60 beats/min and 180 ms acquisition window, with TR=2.6 ms, acquisition over 2 heart beats (2 TEs), slice oversampling (along the partition-encoding dimension, k_{z}) of 10%, ⅝ partial Fourier encoding factor and temporal footprint=55 lines (radial sampling in (kₓ,k_{y}) domain) per slice (i.e., per k_{z}), we can achieve a temporal resolution of 16 heartbeats (˜16 s), which would entail that each stimulation block would take 3 mins (1.5 min each for normo- and hyper-capnic blocks) to acquire 22 whole-heart confounder-reduced T2 maps (constructed from 1260 radial lines). 1.5 min hypercapnic blocks was chosen based on our invasive studies in dogs which showed that (i) peak coronary blood flow due to a hyperemic stimulus is reached in ˜1 min; and (ii) peak hyperemia can be stimulated within 1 min of establishing normocapnia. K-space lines will be acquired throughout the PaCO₂ modulations. Data collected during arrhythmia will not be used in reconstruction. We will collect more data (6 blocks) than needed (from preliminary data) to find the minimum number of blocks required to reach statistical confidence based on GLM analysis (detailed below). PET will be performed before or at end of BOLD.

(b) Reconstruction of Time-Resolved Images and Motion Compensation Computational Framework (FIG. 12)—Motion Estimation, Correction, and Reconstruction of Whole-Heart T2 Maps: To resolve dynamic BOLD signals during free breathing, a motion-correction scheme combined with iterative CS will be used. Respiratory motion correction will be performed in a similar manner with some differences. K-space lines will be sorted into 6 bins, each corresponding to a respiratory state with bin 1 as reference (end-expiration) and the last bin as bin 6 (end inspiration). Based on bin 1, images from bins 2-6 will be registered and transformation parameters will be estimated between the different bins. The k-space data will be again divided into 6-respiratory bins, and the estimated affine transforms will be applied in k-space to the corresponding lines to generate a motion-free dataset. This approach has been validated for accuracy in coronary MRA (vs. navigator) and cardiac T2 mapping (vs. breath-held CMR). To increase temporal resolution, we will use parallel imaging and sparsity in the temporal domain. Specifically, the motion-free dataset will be first separated on the basis of acquisition coils and the corresponding coil sensitivity will be estimated by combining all k-space lines. Then, the k-space lines will be split into two subsets based on TEs. In each subset, every 55 consecutive radial lines will be grouped into continuous time points and reconstructed by iterative radial reconstruction with temporal total-variance (TV) constrains to achieve a 5-fold acceleration with 144×144 in-plane resolution, where the fully sampled k-space requires 245 lines. Temporal TV has been shown to reconstruct images with high SNR105 using even higher acceleration factors. The dynamic image series from each TE will be reconstructed using

$$argmin ( {‖ F \cdot c \cdot d - m ‖}_{2}^{2} + λ \vert T \cdot d {\vert}_{1} ) ,$$

where F is non-uniform fast Fourier transform (NUFFT), c is coil sensitivity, d is the estimated time series of images, m is the measurement, γ is the weight of regularizer, and T is the temporal TV operator. Weighted images will be fitted to a mono-exponential model to derive T2 maps (˜16 s per map).

### Myocardial Segmentation and GLM Data Analysis Framework:

BOLD images will be segmented (to isolate the myocardium) and registered (to correct for cardiac motion between time points) using algorithms that are designed to preserve spatio-temporal changes in intensity due to BOLD effects. Algorithms developed for 2D(+time) processing will be extended to 4D (3D+time).

We estimate that at least 10 data points will be available for each block (normocapnia and hypercapnia) with volumes of 144×144×12 voxels each. A concatenated fully registered 4D dataset for each study will consist of 144×144×12×N voxels, where N=10K; K is the number of blocks (1 to 6).

This 4D dataset will be analyzed by fitting voxel-based supervised (e.g., GLM) and unsupervised (e.g., ICA) models. An in-house tool that we previously developed for segmental analysis will be extended to per-voxel level analysis. This tool will output a P-statistic volume, where for each voxel in the myocardium the p-value of the significance of the relation to the stimulus is reported. We will then threshold the SPM by identifying the voxels that have p<0.05. These voxels will be identified as responders (i.e., responsive to hypercapnia) and will be used to determine relative hyperemic volume (VBOLD), where VBOLD=VH/(total voxels in myocardium) and VH is the total responding voxels with appropriate scaling factors to account for the non-isotropic voxel size, if needed. To validate the accuracy of our approach, we will estimate a similar metric using ¹³N-PET images: images acquired under hypercapnia and normocapnia will be used to derive myocardial perfusion reserve (MPR), corrected for rate-pressure-product (RPP), and the responding pixels will be identified based on MPR>2. Based on total myocardial volume and hyperemic pixels, we will estimate relative hyperemic volume from PET as (V_{PET}) for each K. V_{PET} will be regressed against VBOLD and Bland-Altman plots will be constructed. Minimum K will be identified with ANOVA.

Power: Let a be the standard deviation of median t-scores for a given block. For testing the null hypothesis that the median t-score=3 vs. the alternative hypothesis that it is >3, data from 20 animals achieve 80% power to detect an effect size of 0.66 σ (two-sided t-test, α=0.05), which is practically meaningful.

Acquisition & Reconstruction: We have shown previously that +25-mmHg hypercapnia and clinical dose of adenosine are equivalent. Our goal here is to show that the healthy myocardium can be identified with unprecedented confidence in BOLD CMR within the clinically viable imaging duration using +25-mmHg hypercapnia with ¹³N-PET as the validation standard. Based on our theoretical estimates and our feasibility studies, we expect to generate 3D T2 maps with temporal resolution of 16 heartbeats (˜16 s), which would permit 4 repeat stimulations with at least 8 data points to be acquired in the span of 12 mins (6 min of total hypercapnia, with 4 intermittent hypercapnic periods (1.5 mins each)). Note that even though we aim to minimize hypercapnic duration to the duration of adenosine stress, adenosine is not a candidate for repeat stimulation for many reasons (a key reason is that >70% of people do not tolerate the early reaction to adenosine). GLM Analysis: Using accurate segmentation, registration and GLM, we expect significant responses to the model, along with a single large cluster encompassing the majority of the myocardium (i.e., V_{BOLD}˜1). We also expect to see a direct correlation between V_{BOLD} and V_{PET}, based on our experience with other pixel-based metrics on BOLD. We anticipate that the correlation between V_{BOLD} and V_{PET} to be maximized for K≥4. Although we will acquire significantly more data points than we expect to need (i.e., K=6, total reconstruction time ˜2-3 hrs), we will identify the minimum acceptable K that achieves comparable performance, to minimize hypercapnia and imaging duration.

Potential Difficulties & Alternate Methods: (i) Acquisition & Reconstruction: (A) SNR: While unexpected, if SNR limitations arise, we will first reduce temporal resolution to increase SNR. If further SNR increases are needed, thicker imaging slices (3×3×10 mm³; standard in FPP8) will be used, which can amplify SNR by ˜7-fold. We recently showed that such slices thickness increases do not compromise the capacity to detect and quantify ischemic burden. (B) Nonlinear Filtering: CS can introduce spatial or temporal blurring. If this occurs, we will (i) employ segmental analysis; (ii) optimize CS regularization against ground truth to minimize potential filtering errors; and/or (iii) tradeoff temporal blurring against temporal resolution and SNR. (C) Coil Sensitivity: The motion-correction framework is effective for mitigating respiratory motion and enabling near-perfect respiratory navigator efficiency. However, the coil sensitivity can be different in each respiratory phase and lead to inaccurate sensitivity maps, which may impede the quality of iteratively reconstructed images. However, this has not been shown to be limiting by others. If the image quality is inadequate, a sliding window reconstruction with k-space-weighted image contrast (KWIC) radial filter will be used to achieve time-resolved reconstruction although at the cost of lower temporal resolution and averaging of temporal signals. (D) Arrhythmia Detection: Since HR can increase during hypercapnia, it may be possible that data points from hypercapnia to be misidentified as stemming from arrhythmia. Should this happen, we will only reject data from consecutive data points from erratic changes in HR during the hypercapnic periods. Alternatively, we can introduce additional regressors within the GLM model, to account for such confounders, as typically done in brain fMRI to correct for HR. iii) GLM Analysis: We have adopted a univariate analysis in that we fit and test each voxel independently. This may lead to correlation among neighboring voxels being ignored, hyperemic voxels missed or falsely identified, leading to holes or spurious small clusters in the thresholded SPM maps. These can be partially remedied by smoothing the dataset using 3D Gaussian kernel prior to statistical analysis, a standard practice in fMRI to increase voxel-to-voxel correlation. Multivariate approaches (e.g., ICA with spatial constraints) would be adopted if these methods underestimate VBOLD.

### Example 2-2. To Test and Validate in a Large Animal Model of Clinically Significant Coronary Artery Stenosis that the Proposed CMR Approach can Enable Accurate Determination of Ischemic Burden

We will use the method developed in Example 2-1 to show that it is possible to accurately delineate the ischemic myocardium in canines with significant coronary stenosis of varying extent. The endpoint of this Example 2-2 is to determine the minimum number of hypercapnic blocks that would enable an assessment of the ischemic burden as accurately as ¹³N-PET within total hypercapnia exposure of <6 mins.

In Example 1, we tested whether repeat hypercapnic stimulations can improve the confidence in identifying the ischemic territories based on BOLD CMR. Using the HR-independent 3D T2 CMR (prelim. data from Example 2-1), we tested the relationship between statistical confidence and repeat hypercapnic modulation (+25 mmHg). Canines (n=7) with a non-flow limiting coronary stenosis (fractional flow reserve (FFR) of ˜0.75) were studied in a clinical PET/MR scanner with ¹³N PET for validation. With each additional stimulation, the confidence for identifying the healthy myocardium increased, which enabled greater accuracy for identifying the ischemic regions as the complement. Both ischemic and healthy myocardial territories identified on segmental SPMs derived from BOLD CMR were highly concordant with respective territories on ¹³N-PET. Hence, repeated stimulation of hearts with intermittent hypercapnia and whole 3D T2 CMR increased sensitivity, specificity and accuracy of BOLD CMR to >90% at FFR˜0.75.

However, this approach takes 24 mins of exposure to hypercapnia. In this Example 2-2 we will test whether the method developed in Example 2-1 can reliably detect clinically significant IHD (i.e., across various controlled FFR) within a hypercapnia exposure of <6 mins. Methods:

Data Collection and Analysis: Animals will be implanted with hydraulic LAD occluders and aortic pressure catheters (Vertebrate Animals). On the day of the imaging, they will be additionally instrumented with MRI-compatible 2F pressure catheters distal to the occluder to establish different levels of FFR across stenoses.

1. Time-resolved BOLD CMR (as shown in Example 2-1) will be prescribed at various FFR values (1.0, 0.9, 0.75, and 0.50) with K=6. FFR values will be established by adjusting the hydraulic occluder under hypercapnia (+25 mmHg above baseline). ¹³N-PET will be performed before or after BOLD CMR scans at each FFR at normocapnia and hypercapnia with sufficient time to permit PET tracer decay.

2. 4D BOLD (3D+t) image series acquired under PaCO₂ changes will be used to derive SPM.

3. The voxels that do not respond to ΔPaCO₂ (on SPM) will be identified as ischemic. Ischemic burden will be quantified as V_{iBOLD}=1−V_{H}/total myocardial voxels, where V_{H} is as defined in Example 2-1 and regressed against FFR. ANOVA will be used to identify the minimum K needed to accurately quantify ischemic burden (with PET as ground truth). Paired t-test will be used to test if ViBOLD (FFR=0.75)>ViBOLD (FFR=1.0).

4. Detection of Ischemic Territories (validation): To validate that the ischemic territories are accurately identified, a visual analysis of AHA segments of BOLD and PET images based on ASNC guidelines will be performed. Wilcoxon signed-rank test will be used to compare the visual scores between BOLD and PET images. Quantification of Ischemic Burden (validation): ¹³N-PET MPR at each FFR will be used to compute relative perfusion defect volumes (V_{iPET}), similar to V_{iBOLD}. V_{iBOLD} and V_{iPET} will be regressed.

Power: 30 animals can achieve 80% power to detect an effect size of 0.53 σ_{d} (2-sided paired t-test, α=0.05), where σ_{d} is the SD of the difference in the mean t-scores between remote and affected regions for a given number of stimulations (this is conservative).

Data Interpretation: we expect that repeat hypercapnia with K≥3 would enable accurate identification of ischemic territories as the BOLD response in stenotic territories will be reduced. We also expect that these territories will not respond to hypercapnia and will have large p values in SPM. Since stenosis severity is related to the extent of myocardial territory affected29, we expect to find a direct relationship between V_{iBOLD} and FFR. Hence, V_{iBOLD} (FFR=0.75) should be significantly greater than V_{iBOLD} (FFR=1.0). Also based on preliminary data, we expect to find significant correlation between myocardial territories visually identified to be ischemic on SPM and ¹³N-PET. Similarly, we expect to find that the ischemic volume (burden) of the myocardium, determined from SPMs and ¹³N-PET to be highly correlated.

Potential Difficulties & Alternate Methods: (A) If some voxels are misidentified as ischemic, we will use multivariate analysis to identify optimal clusters of voxels. (B) We may find that the minimum K for identifying ischemic myocardium on SPMs is lower for smaller FFR due to coronary steal. If so, we will consider the minimum value of K that permits identification of ischemic myocardium as accurately as PET for FFR of 0.75 as the optimal K. Limitations: (A) Ischemic burden will be identified as the total myocardial volume with MPR<2 (definition of clinically significant ischemic territory). Additional thresholds of MPR to further discretize severity of ischemia may be possible, but this will be tested once we establish that SPMs can identify all ischemic territories. (B) The proposed approach is tested in one-vessel stenosis affecting the proximal segment of LAD. Additional efforts will be needed to extend to multi-vessel or microvascular disease. Such efforts need to take into account the distribution of non-responsive voxels and their preferential clustering in the sub-endocardium.

### Example 2-3. To Show in Patients with Clinically Significant IHD that the Proposed cfMRI Approach can Quantify Ischemic Burden with Safety, Tolerability and Accuracy that is not Different From ¹³N-PET with Adenosine Stress

Rationale: RESPIRACT allows precise control of PaCO₂, within 1 mmHg, under constant O₂. It permits rapid PaCO₂ changes (1-2 breaths) that are independent of minute ventilation. More than 4000 patient studies (age 4-72) have used RESPIRACT worldwide with little or no complications. In Task 1 we will evaluate the safety of hypercapnia in IHD patients. In Task 2 we will evaluate the diagnostic accuracy of the proposed cfMRI approach in patients with single-territory IHD against ¹³N-PET and their comparative tolerance to PaCO₂ modulation vs. adenosine. These studies will provide the first evidence into the safety, tolerability and diagnostic accuracy of the proposed cfMRI approach in IHD patients.

Hypercapnia induces myocardial BOLD and blood flow response in healthy volunteers. We studied healthy volunteers (n=18) at baseline and under various hypercapnia levels (ΔPaCO₂ of +5 to +20 mmHg). We acquired 2D T2-weighted BOLD CMR and PET under repeat hypercapnia to assess reproducibility. Myocardial hyperemia was evident in BOLD images. BOLD response and PET-MPR (myocardial perfusion reserve) were linearly correlated with PaCO₂ (R=0.7). ΔPaCO₂ of +15 mmHg invoked a MPR >2 reproducibly at repeated stimulation. Hence, in humans (i) hypercapnia mediates a BOLD response; (ii) ΔPaCO₂≥15 mmHg can lead to PET-MPR>2; and (iii) repeat hypercapnic stimulation can reproduce the MPR. We will build on these findings in IHD patients (using methods from Example 2-1) using RESPIRACT technology to reliably detect IHD with BOLD CMR, and validate our findings against ¹³N-PET.

Human tolerability to hypercapnia is not different from adenosine. Healthy subjects (n=10; age: 33-72), randomized to adenosine and hypercapnia (prescribed for 6 mins), were studied in a MR scanner. Subjects scored their tolerability [1 (no discomfort) to 10 (intolerable)] after each test. Tolerance for adenosine and hypercapnia were not different (p=0.4); but hypercapnia was preferred over adenosine (n=7 vs n=3). Hence, hypercapnia of 25 mmHg is at least as tolerable as adenosine. We build on these in patients with IHD.

Early evidence showed hypercapnia is safe in patients with IHD. IHD patients with greater than 15% of ischemic mass from SPECT (age: 52-67; 4 male; 1 female), while supine, were exposed to repeated (four) exposure to 2-minutes of 25 mmHg of hypercapnia with RESPIRACT. Patient vitals (ECG, SPO2 and blood pressure) were monitored by a cardiologist. All patients tolerated intermittent hypercapnia; 2 patients complained of mild (n=1) or moderate discomfort (n=1), but no safety concerns were observed. Hence hypercapnia of 25 mmHg appears safe in IHD patients.

### Methods:

Inclusion criteria: patients (a) ≥18 years of age; (b) no prior MI (based on clinical history); (c) with single-territory ischemia on SPECT or PET before diagnostic coronary angiography; and (d) informed consent;

Exclusion Criteria: (a) acute coronary syndrome, acute myocardial infarction, ongoing myocardial ischemia, history of coronary artery bypass grafting, unstable angina pectoris, and coronary artery interventions in the time between PET/SPECT and BOLD CMR studies, contraindications for adenosine and contrast media and severe arrhythmias; (b) any cardiac/general medical condition precluding the completion of a CMR study (e.g., heart failure, claustrophobia, implanted device non-MR compatible); (c) prior history of non-ischemic cardiomyopathy (NICM) or more than moderate valvular disease; and (d) GFR <45 ml/min; Recruitment and Consent: Dr. Berman (Co-I) will identify candidates with stable chest pain and suspected IHD scheduled for a clinically indicated MBF imaging at Cedars and will discuss the study. Following written informed consent, patients will be recruited for the study. Cedars-Sinai performs 5-7 ischemia tests per day;

Collection: ECG and non-invasive blood pressure will be monitored throughout the study. Blood gases will be monitored with RESPIRACT.

Task 1: Each patient, while supine, will undergo hypercapnia of 25 mmHg followed by normocapnia in a patient preparation room. Changes in PaCO₂ will be imposed every 2 minutes. Patient vitals (ECG, blood pressure and SPO2) as well as discomfort, chest pain, dyspnea, high-grade AV-block and bronchospasm or other life-threatening adverse events will be recorded. Test will be discontinued if medically indicated or if the patient wishes to discontinue.

Task 2: Proposed BOLD CMR approach will be prescribed in a clinical PET/MR system under PaCO₂ modulation with the minimum K identified in Example 2-2. At the end of the BOLD CMR, ¹³N-PET with adenosine will be acquired. At the end of the exam the patients will be asked to score the tolerability [1 (no discomfort) to 10 (intolerable)] during cfMRI and adenosine stress.

Data Analysis: Task 1: In patients whom PaCO₂ modulation is discontinued will not undergo Task 2. Adverse effects, the frequency of occurrence and the number of subjects in whom PaCO₂ modulation was discontinued will be evaluated and reported. Task 2: BOLD SPM maps will be constructed as in Example 2-2. Two expert CMR readers (blinded to clinical history) will analyze images as in Example 2-2 for presence and location using a 17-segment model. The presence and location of ischemia will be scored between 0-4 (scale: absent (0) to certain (4)). The ischemic burden will be computed as in Example 2-2. Visual and quantitative assessments based on SPM BOLD will be regressed against ¹³N-PET and Bland-Altman plots will be constructed. The Kappa (κ) measure of agreement (and 95% confidence interval) will be calculated. The tolerance to hypercapnic stimulus and adenosine will be compared using paired t-tests.

Power: The sample size is determined to achieve >80% power for non-inferiority of the proposed method and precision of the estimate of agreement between the two raters. Based on prior studies (agreement between blinded readers for ischemia on PET is 87%), we estimate the SD of Cohen's κ measure of inter-rater agreement to be between 0.25 and 0.28. Hence, data from 68 to 85 patients result in a two-sided 95% confidence interval to estimate κ to within 0.06 margin of error. Using a sample size of 85 patients, a non-inferiority test with a margin of equivalence of 0.27SD will achieve 80% power to detect a non-inferiority using a one-sided t-test with α=0.05. The margin of equivalence equals to ⅓ of the SD, which is practically significant. Assuming a 10% dropout we will study 94 patients as part of this Example 2-3.

Expected Results and Interpretation: Task 1: Based on previous studies in non-IHD patients, our studies in healthy humans and pilot studies in IHD patients, no safety concerns are anticipated. Task 2: Based on our findings in canines and humans to date, we expect the proposed method to permit detection of ischemia as accurately as ¹³N-PET. Based on preliminary data from Example 2-2, we expect to find that visual scoring for the presence and location of ischemic zones on BOLD SPMs with the proposed approach and PET to be tightly correlated. Similarly, we expect to find that the relative perfusion defect identified on BOLD SPM is closely correlated with R2>0.9 and bias <5%. Based on preliminary data, we anticipate the tolerance of IHD patients to intermittent hypercapnia to be not different from adenosine. The endpoint of this Example 2-3 is to show in IHD patients, the safety, tolerance and diagnostic accuracy of ischemic burden based on the proposed cfMRI method are not inferior to ¹³N-PET with adenosine within a margin of equivalence of <0.5 SD.

Potential Difficulties and Alternate Approaches:

Task 1: In the unlikely event safety concerns become evident, studies will be immediately terminated, patients will be exposed to room air to re-establish baseline PaCO₂ and treated as needed.

Task 2: (A) Given the resolution differences between PET and MR, sub-endocardial defects not identified in PET may be identified in cfMRI. If this happens, we will down-sample cfMRI resolution to match that of PET and re-analyze the data to remove resolution bias. (B) Some patients may refuse adenosine stress after hypercapnia. If our 10% dropout estimate cannot support this, we will recruit additional patients. (C) While not anticipated, if the tolerance to hypercapnia is poor, we will explore known remedies to improve tolerance (e.g., pre-treatment with Tylenol 3121, weaker hypercapnia at the cost of increased scan time).

To provide aspects of the present disclosure, embodiments may employ any number of programmable processing devices that execute software or stored instructions. Physical processors and/or machines employed by embodiments of the present disclosure for any processing or evaluation may include one or more networked (Internet, cloud, WAN, LAN, satellite, wired or wireless (RF, cellular, WiFi, Bluetooth, etc.)) or non-networked general purpose computer systems, microprocessors, filed programmable gate arrays (FPGAs), digital signal processors (DSPs), micro-controllers, smart devices (e.g., smart phones), computer tablets, handheld computers, and the like, programmed according to the teachings of the exemplary embodiments. In addition, the devices and subsystems of the exemplary embodiments can be implemented by the preparation of application-specific integrated circuits (ASICs) or by interconnecting an appropriate network of conventional component circuits. Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, database management software, and the like. Computer code devices of the exemplary embodiments can include any suitable interpretable or executable code mechanism, including but not limited to scripts, interpretable programs, dynamic link libraries (DLLs), Java classes and applets, complete executable programs, and the like. Moreover, processing capabilities may be distributed across multiple processors for better performance, reliability, cost, or other benefits.

Common forms of computer-readable media may include, for example, a floppy disk, a flexible disk, a hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read. Such storage media can also be employed to store other types of data, e.g., data organized in a database, for access, processing, and communication by the processing devices.

Hence, further embodiments of the invention provide computer readable storage media containing computer executable instructions for a method comprising: (a) directing an MRI system to perform a first sequence that is sensitive to blood oxygenation, blood volume, and/or blood flow, at a cardiovascular system of a subject at a reference partial pressure of arterial carbon dioxide (PaCO₂) level to acquire a first set of reference MR data from the subject; (b) directing a gas delivery system to administer an effective amount of a stress agent selected from the group consisting of CO₂ and an admixture comprising CO₂ to attain a stress PaCO₂ level in the subject; (c) directing the MRI system to perform a second sequence that is sensitive to the blood oxygenation, the blood volume, and/or the blood flow at the cardiovascular system of the subject at the stress PaCO₂ to acquire a first set of stress MR data from the subject; (d) repeating steps (a)-(c) at least once, each repetition generating a subsequent set of reference MR data and a subsequent set of stress MR data; (e) reconstructing a series of reference images from each set of the reference MR data and registering the series of reference images to obtain a motion-corrected reference image for each set of the reference MR data, thereby obtaining a plurality of motion-corrected reference images from repetition of step (a), and reconstructing a series of stress images from each set of the stress MR data and registering the series of stress images to obtain a motion-corrected stress image for each set of the stress MR data, thereby obtaining a plurality of motion-corrected stress images from repetition of steps (b) and (c).

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method for performing a cardiac stress testing, detecting the presence of or determining the progression of a cardiovascular disease, and/or assessing the risk of developing the cardiovascular disease in a subject, using a magnetic resonance imaging (MRI) system, the method comprising:
(a) administering a stress agent to the subject over one or more periods of time in one or more amounts, wherein at least one amount is effective for increasing blood velocity and/or flow rate at the cardiovascular system of the subject;
(b) directing the MRI system to perform a sequence that is sensitive to blood oxygenation, blood volume, and/or blood flow at the cardiovascular system of the subject to acquire a plurality of MR data sets corresponding to a plurality of MR acquisitions,
wherein the plurality of MR acquisitions comprises one or more acquisitions whose MR data set is acquired during the one or more periods of time when the at least one effective amount of the stress agent is administered to the subject, and one or more acquisitions whose MR data set is acquired when the subject is at rest or during administration of a different amount of the stress agent to the subject;
(c) reconstructing a series of images from each MR data set and registering the series of images to obtain a motion-corrected image for each MR acquisition, thereby obtaining a plurality of motion-corrected images corresponding to the plurality of MR acquisitions; and
(d) comparing the plurality of motion-corrected images in terms of image voxels or pixels characteristic of blood oxygenation, blood volume, or blood flow in at least one region of the cardiovascular system,
wherein an absence of a statistically significant difference in the image voxels or pixels in at least one region of the cardiovascular system compared among the motion-corrected images acquired during the administration of the effective amount of the stress agent and those acquired when the subject is at rest or during the administration of the different amount of the stress agent is indicative of impaired blood oxygenation, impaired blood volume, or impaired blood flow, respectively, in the at least one region of the cardiovascular system of the subject.

2. The method of claim 1, wherein the number of acquisitions in the plurality of MR acquisitions is sufficient for a statistical power of 0.8 or greater in the comparing step, in terms of a null hypothesis (H₀) that the stress agent has no effect on the blood volume, the blood flow, or the blood oxygenation in the at least one region of the cardiovascular system of the subject.

3. The method of claim 1, wherein:
step (a) includes administering to the subject an effective amount of the stress agent for increasing blood velocity and/or flow rate in the subject in two or more periods of time;
step (b) includes directing the MRI system to perform the sequence to acquire a MR data set during each of the periods when the stress agent is administered according to step (a), and to acquire one or more MR data sets when the subject is at rest or when a baseline level of the stress agent is administered, thereby obtaining a plurality of MR data sets corresponding to a plurality of MR acquisitions;
and step (d) includes comparing the motion-corrected images acquired in the two or more periods when the effective amount of the stress agent is administered with the motion-corrected images acquired when the subject is at rest or when a baseline level of the stress agent is administered, in terms of image voxels or pixels characteristic of the blood oxygenation, the blood volume, or the blood flow in the at least one region of the cardiovascular system, wherein an absence of a statistically significant difference in the image voxels or pixels between the motion-corrected images acquired in the two or more periods when the effective amount of the stress agent is administered and the motion-corrected images acquired when the subject is at rest or when a baseline level of the stress agent is administered is indicative of impaired blood oxygenation, impaired blood volume, or impaired blood flow, respectively, in the at least one region of the cardiovascular system of the subject.

4. The method of claim 1, wherein
step (a) includes administering the stress agent in a stepwise manner so as to attain an incremental partial pressure of arterial carbon dioxide (PaCO₂) level over the one or more periods of time in the subject; step (b) includes directing the MRI system to perform the sequence to acquire a MR data set when the subject is at each PaCO₂ level following an increment of the administered stress agent, thereby obtaining a plurality of MR data sets corresponding to a plurality of MR acquisitions when the subject is at at least two different levels of PaCO₂;
and step (d) includes comparing the plurality of motion-corrected images acquired at the at least two different levels of PaCO₂, wherein an absence of a statistically significant difference in image voxels or pixels in at least one region of the cardiovascular system compared among the motion-corrected images acquired at the at least two different levels of PaCO₂ in indicative of impaired blood oxygenation, impaired blood volume, or impaired blood flow, respectively, in the at least one region of the cardiovascular system of the subject.

5. The method of claim 1, wherein the sequence comprises a pulse for motion control and a pulse for confounder correction in a three-dimensional MRI acquisition.

6. The method of claim 1, wherein the reconstructing step comprises compressed sensing reconstruction.

7. The method of claim 1, wherein the sequence comprises a preparation pulse and a readout pulse, wherein the preparation pulse comprises a saturation recovery (SR) pulse, or a SR pulse in combination with a navigator pulse and/or an adiabatic pulse, and wherein the readout pulse comprises a spoiled gradient-echo (GRE) pulse and/or a balanced steady-state free precessing (bSSFP) sequence.

8. The method of claim 7, wherein the saturation recovery pulse has a constant saturation recovery time.

9. The method of claim 1, further comprising segmenting the cardiovascular system in the plurality of the motion-corrected images, and wherein the step (d) includes comparing at least a same segment of the cardiovascular system among the plurality of the motion-corrected images, wherein an absence of a statistically significant difference in the image voxels or pixels in the same segment compared among the motion-corrected images acquired during the administration of the effective amount of the stress agent and those acquired when the subject is at rest or during the administration of the different amount of the stress agent is indicative of impaired blood oxygenation, impaired blood volume, or impaired blood flow, respectively, in at least the same segment of the cardiovascular system of the subject.

10. The method of claim 9, wherein the segmentation includes one segment of endocardium and one segment of epicardium, wherein the absence of a statistically significant difference in the image voxels or pixels in the endocardium segment among the motion-corrected images and the absence of a statistically significant difference in the image voxels or pixels in the epicardium segment among the motion-corrected images is indicative of balanced myocardial ischemia in the subject.

11. The method of claim 1, wherein the sequence is based on one or more contrasts created from T1, T2, T2*, and arterial spin labeling (ASL).

12. The method of claim 1, wherein the stress agent comprises CO₂, regadenoson, adenosine, dipyridamole, dobutamine, or a combination thereof.

13. The method of claim 1, wherein the stress agent is selected from the group consisting of CO₂ and an admixture comprising CO₂, and administered via inhalation by the subject.

14. The method of claim 1, wherein the administering of the stress agent comprises administering CO₂ or an admixture comprising CO₂ so as to change the PaCO₂ of the subject in a range of 20 mmHg to 80 mmHg PaCO₂, 30 mmHg to 80 mmHg PaCO₂, 40 mmHg to 80 mmHg PaCO₂, 50 mmHg to 80 mmHg PaCO₂, 60 mmHg to 80 mmHg PaCO₂, 70 mmHg to 80 mmHg PaCO₂, 20 mmHg to 70 mmHg PaCO₂, 30 mmHg to 70 mmHg PaCO₂, 40 mmHg to 70 mmHg PaCO₂, 50 mmHg to 70 mmHg PaCO₂, 60 mm Hg to 70 mmHg PaCO₂, 20 mmHg to 60 mmHg PaCO₂, 30 mmHg to 60 mmHg PaCO₂, 40 mm Hg to 60 mmHg PaCO₂, or 50 mmHg to 60 mmHg PaCO₂.

15. The method of claim 4, wherein the stress agent is administered in a stepwise manner so as to attain a PaCO₂ level in the subject in increments of about 5 mm Hg, 10 mm Hg, 15 mm Hg, 20 mm Hg, or 25 mm Hg.

16. The method of claim 1, wherein the cardiovascular disease comprises one or more of infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, coronary stenosis, LAD stenosis, and peripheral artery disease.

17. The method of claim 1, wherein the cardiovascular disease is ischemic heart disease, and the cardiovascular system comprises a myocardium and/or a coronary artery.

18. The method of claim 1, wherein the subject at rest is when no stress agent is administered to the subject, and the subject at rest has a baseline PaCO₂ level measured at the end tidal phase.

19. The method of claim 1, further comprising conducting a pre-scan with the subject, wherein the pre-scan comprises administering a first test amount of the stress agent to the subject and directing the MRI system to acquire a first test MR data set, wherein if the subject's response to the first test amount is tolerable and/or effective, or adjusting the first test amount to a second test amount if the subject's response to the first test is unsafe or ineffective, and repeating the pre-scan test until the subject's response in the pre-scan test is tolerable and/or effective.

20. The method of claim 1, further comprising selecting, providing, and/or administering a therapy for treating and/or preventing the cardiovascular disease for the subject.

21-24. (canceled)
